SEQUENCE LISTING (22 sequences)

SEQ ID NO: 1 - protein, 38 aa, Artificial Sequence
  Description of Artificial Sequence Synthetic peptide
MKFNKKRVAIATFIALIFVSFFTISSIQDNQANAAERS

SEQ ID NO: 2 - protein, 32 aa, Artificial Sequence
  Description of Artificial Sequence Synthetic peptide
MKFNKKRVAIATFIALIFVSFFTISSIQDAQA

SEQ ID NO: 3 - protein, 36 aa, Artificial Sequence
  Description of Artificial Sequence Synthetic peptide
MKFNKKRVAIATFIALIFVSFFTISSIQDAQAAERS

SEQ ID NO: 4 - protein, 35 aa, Artificial Sequence
  Description of Artificial Sequence Synthetic peptide
MKFNKKRVAIATFIALIFVSFFTIIPNTAQAAERS

SEQ ID NO: 5 - protein, 36 aa, Artificial Sequence
  Description of Artificial Sequence Synthetic peptide
MKFNKKRVAIATFIALIFVSFFTISSIQDAQADTRS

SEQ ID NO: 6 - protein, 35 aa, Artificial Sequence
  Description of Artificial Sequence Synthetic peptide
MKNKKRVAIATFIALIFVSFFTISSIQDAQAAERS

SEQ ID NO: 7 - protein, 35 aa, Artificial Sequence
  Description of Artificial Sequence Synthetic peptide
MKFKKRVAIATFIALIFVSFFTISSIQDAQAAERS

SEQ ID NO: 8 - protein, 36 aa, Artificial Sequence; identical to SEQ ID NO: 5
  Description of Artificial Sequence Synthetic peptide

SEQ ID NO: 9 - protein, 35 aa, Artificial Sequence
  Description of Artificial Sequence Synthetic peptide
MKFNKKRVAIATFIALIFVSFFTISSIDAQAAERS

SEQ ID NO: 10 - protein, 38 aa, Artificial Sequence
  Description of Artificial Sequence Synthetic peptide
MKFNKKRLLLLLLLLLLLLLLLLISSIQDNQTNAAERS

SEQ ID NO: 11 - DNA, 752 bp, Homo sapiens
  CDS (3)..(740)
gctcttccgcagatgaaccaccacaatcaccatgggatcgtgttaaagatcttgctacagtttatgttgatgttcttaaagattcaggtcgtgattatgtttcacaatttgaaggttcagctcttggtaaacaacttaatcttaaacttcttgataattgggattcagttacatcaacattttcaaaacttcgtgaacaacttggtccagttacacaagaattttgggataatcttgaaaaagaaacagaaggacttcgtcaagaaatgtctaaagatcttgaagaagttaaagcaaaagttcaaccatatcttgatgattttcagaaaaaatggcaagaagaaatggaattgtatcgtcaaaaagttgaaccacttcgtgctgaacttcaagaaggtgctcgtcaaaaacttcatgaattacaagaaaaactttcaccacttggagaagaaatgcgtgatcgtgctcgtgctcatgttgatgctttacgtacacatcttgctccatattcagatgaacttcgtcaacgtcttgctgctcgtcttgaagctcttaaagaaaatggaggagcacgtcttgctgaatatcatgctaaagctacagaacatctttcaacactttcagaaaaagctaaaccagctcttgaagatcttcgtcaaggtcttttgccagttcttgaatcatttaaagtttcttttctttcagctttggaagaatatacaaaaaaacttaatacacaataataagtcgac

SEQ ID NO: 12 - DNA, 48 bp, Artificial Sequence
  Description of Artificial Sequence Synthetic oligonucleotide
gatgctcaagcagccgaaagatctgatatcactagtctgcaggtcgac

SEQ ID NO: 13 - protein, 8 aa, Artificial Sequence
  Description of Artificial Sequence Synthetic peptide
DAQAAERS

SEQ ID NO: 14 - DNA, 24 bp, Artificial Sequence
  Description of Artificial Sequence Synthetic oligonucleotide
gatgctcaagcagccgaaagatct

SEQ ID NO: 15 - protein, 4 aa, Artificial Sequence
  Description of Artificial Sequence Synthetic peptide
AERS

SEQ ID NO: 16 - DNA, 32 bp, Artificial Sequence
  Description of Artificial Sequence Synthetic oligonucleotide
gatgctcaagcaggaagagcagatctctgcag

SEQ ID NO: 17 - protein, 4 aa, Artificial Sequence
  Description of Artificial Sequence Synthetic peptide
DAQA

SEQ ID NO: 18 - DNA, 17 bp, Artificial Sequence
  Description of Artificial Sequence Synthetic oligonucleotide
ctcgatgctcttccgca

SEQ ID NO: 19 - DNA, 12 bp, Artificial Sequence
  Description of Artificial Sequence Synthetic oligonucleotide
gatgctcaagca

SEQ ID NO: 20 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 21 - protein, 246 aa, Homo sapiens
SSADEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 22 - protein, 7 aa, Artificial Sequence
  Description of Artificial Sequence Synthetic peptide
DAQAXXX

(19) United States
(12) Patent Application Publication
(10) Pub. No.: US 2008/0293102 A1
(43) Pub. Date: Nov. 27, 2008

(54) COMPOSITIONS AND METHODS FOR PRODUCING APOLIPOPROTEIN

(75) Inventors: Maritza OXENDER, Ann Arbor, MI (US); Jean-Louis Dasseux, Toulouse (FR)

Correspondence Address: DECHERT LLP, P.O. BOX 390460, MOUNTAIN VIEW, CA, 94039-0460, US

(73) Assignee: Cerenis Therapeutics Holding, S.A., Labege cedex (FR)

(21) Appl. No.: 12/039,385

(22) Filed: Feb. 28, 2008

Related U.S. Application Data
(60) Provisional application No. 60/892,244, filed on Feb. 28, 2007.

(51) Int. Cl. C12P 21/02; C12N 15/12; C12N 15/74; C12N 15/75; C12N 15/77; C12N 1/21

(52) U.S. Cl. 435/69.1; 536/23.5; 435/320.1; 435/252.31; 435/252.3; 435/252.32

(57) ABSTRACT

The disclosure relates to recombinant nucleic acids, expression vectors comprising the recombinant nucleic acids, and host cells comprising the expression vectors for expressing a protein of interest.

### 1. CROSS REFERENCES TO RELATED APPLICATIONS

This application claims benefit under 35 U.S.C. § 119(e) to U.S. application Ser. No. 60/892,244, filed Feb. 28, 2007, the contents of which are incorporated herein by reference.

### 2. BACKGROUND

Circulating cholesterol is carried by two major cholesterol carriers, low density lipoproteins (LDL) and high density lipoproteins (HDL). LDL appears to be responsible for the delivery of cholesterol from the liver, where it is synthesized or obtained from dietary sources, to extrahepatic tissues in the body. It is believed that plasma HDL particles play a major role in cholesterol regulation, acting as scavengers of tissue cholesterol.

Atherosclerosis is a progressive disease characterized by the accumulation of cholesterol within the arterial wall. The lipids deposited in atherosclerotic lesions are derived primarily from plasma LDL; thus, LDLs are described as the “bad” cholesterol. In contrast, HDL serum levels correlate inversely with coronary heart disease, and as a consequence, high serum levels of HDL are regarded as a negative risk factor. Thus, HDLs are described as the “good” cholesterol.

Recent studies of the protective mechanism(s) of HDL have focused on apolipoprotein A-I (ApoA-I), the major component of HDL. High plasma levels of ApoA-I are associated with absence or reduction of coronary lesions (Maciejko et al., 1983, N Engl J Med. 309:385-89; Sedlis et al., 1986, Circulation 73:978-84). However, the therapeutic use of ApoA-I and known variants of ApoA-I, as well as reconstituted HDL, is limited by the large amount of apolipoprotein required for therapeutic administration and by the cost of protein production, considering the low overall yield of production. Thus, there is a need to develop alternative methods for the production of ApoA-I that can be used to treat and/or prevent cholesterol accumulation within coronary arteries.

### 3. SUMMARY

The present disclosure provides methods and compositions for producing apolipoprotein in secreted form. Apolipoproteins produced according to the descriptions herein find uses as therapeutic agents for treating disorders and diseases associated with lipid metabolism. In some aspects, the disclosure provides recombinant nucleic acids comprising a first polynucleotide encoding a signal peptide and a second polynucleotide encoding an apolipoprotein, where the first and second polynucleotide are operatively linked to direct expression and secretion of the apolipoprotein from the host cell. In some embodiments, the signal peptide comprises the structure

(n)ₓ˜(m)_{y}˜(c)_{z},

wherein
each n is independently any amino acid, with the proviso that two or more n is a basic amino acid residue.
each m is independently an aromatic, aliphatic, hydrophobic or hydroxyl containing amino acid residue;
each c is independently any amino acid, with the proviso that two or more c is a polar amino acid residue;
x is 6, 7, or 8;
y is any integer from 13 to 16; and
z is an integer from 5 to 14;
“˜” is a peptide bond.

In some embodiments, the signal peptide comprises a polypeptide of residues X¹ to X³⁸ having the above structure, and having homology to a signal peptide selected from the group consisting of SEQ ID NOS:1-10. In some embodiments, the encoded signal peptide has at least 60%, at least 70%, at least 80%, at least 85%, at least 90%, at least 95%, at least 96%, at least 97%, at least 98%, or at least 99% or more sequence identity as compared to a reference sequence selected from the group consisting of SEQ ID NOS:1-10. In some embodiments, the reference sequence selected is SEQ ID NO:1, 2 or 10. In some embodiments, the signal peptide sequence for directing secretion of the apolipoprotein is selected from the group consisting of SEQ ID NOS:1-10.

In various embodiments, the signal peptide can comprise modified signal peptides of a reference sequence, where the modifications include substitutions and deletions of amino acid residues in the reference sequence. In some embodiments, the modifications include insertions of amino acid residues into the reference sequence. The corresponding residue positions in the signal peptide that can be modified are described in the detailed description below.

In various embodiments, the second polynucleotide can encode any apolipoprotein or an apolipoprotein mimic or analog. In some embodiments, the second polynucleotide sequence encodes a human apolipoprotein selected from the group consisting of preproapolipoprotein, preproApoA I, proApoA I, ApoA I, preproApoA II, proApoA II, ApoA II, preproApoA-IV, proApoA IV, ApoA IV, ApoA V, preproApoE, proApoE, ApoE, preproApoA IMilano, proApoA IMilano, ApoA IMilano, preproApoA IParis, proApoA IParis, and ApoA IParis. In some embodiments, the polynucleotide encoding the apolipoprotein is codon optimized for expression in a suitable host cell.

The present disclosure further provides expression vectors comprising the recombinant nucleic acids operably linked to one or more control sequences, where the control sequences include, among others, promoters, ribosome bindings sites, and transcription and translation termination sequences. In some embodiments, the expression vectors can further comprise replication origins, integration sequences, and selection markers.

Host cells comprising the recombinant nucleic acids and expression vector can be prepared to express the apolipoproteins. In some embodiments, the host cell is a Gram-positive bacteria. In some embodiments, the Gram-positive bacterium is a lactic acid bacterium. Suitable lactic acid bacteria host cells include, among others, Lactococcus spp., Streptococcus spp., Lactobacillus spp., Leuconostoc spp., Pediococcus spp., Brevibacterium spp. and Propionibacterium spp. In some embodiments, the host cell used to express the apolipoprotein is deficient in various intracellular and/or extracellular proteases to limit undesirable proteolytic processing of the expressed polypeptide. In some embodiments, the host cells are deficient in the proteases represented by HtrA and/or PrtP.

Once made, the host cells can be used in methods to produce apolipoprotein in secreted form. In some embodiments, the method comprises culturing the host cell under conditions suitable for expression of the encoded apolipoprotein. The culturing medium can be chemically defined or undefined medium. In some embodiments, the culture medium can comprise a liquid medium, which allows rapid separation of the cells from the apolipoprotein secreted into the medium. The apolipoprotein can be isolated away from the cells by any number of techniques, such as filtration, centrifugation, and electrophoresis.

In some embodiments, the culture medium is treated to remove components that affect host cell growth. For instance, where the host cell is a lactic acid bacterium, accumulation of lactic acid can slow cell growth and limit protein expression. Thus, in some embodiments, the lactic acid can be removed by various techniques, such as chromatography or electro-enhanced dialysis, to enhance production of apolipoprotein under the culture conditions.

Apolipoproteins produced using the compositions and methods described herein can be used in a variety of applications, including its use in forming apolipoprotein-lipid complexes or apolipoprotein-phospholipid complexes for treating various lipid associated disorders, such as coronary heart disease; coronary artery disease; cardiovascular disease; hypertension; restenosis; vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein. Other uses of the expressed apolipoproteins will be apparent to the skilled artisan.

### 4. BRIEF DESCRIPTION OF THE FIGURES

FIG. 1 provides the sequences of signal peptides, SEQ ID NOS:1-10, for producing secreted apolipoprotein;

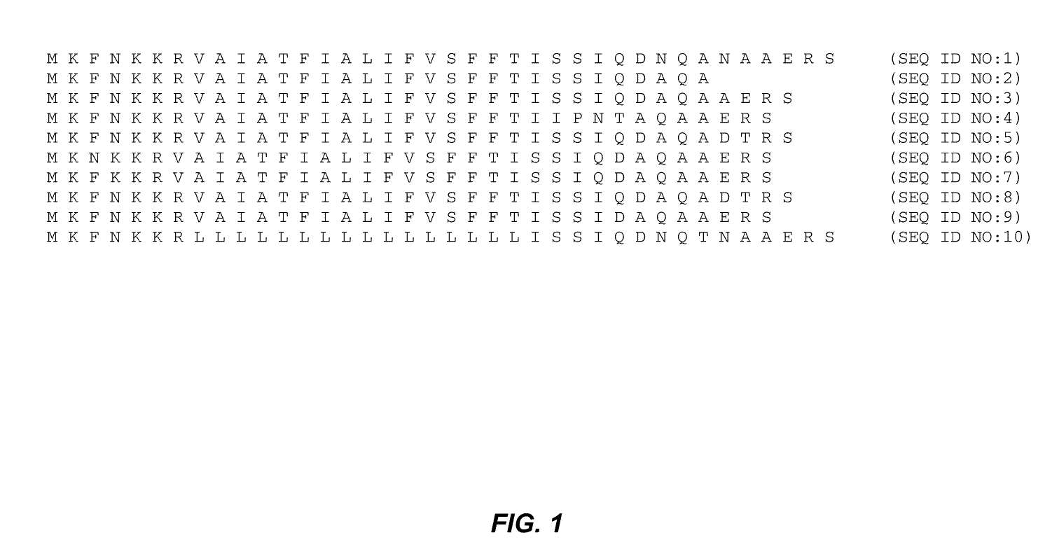

FIG. 2 shows the signal peptidase cleavage site in two signal peptides, one construct in which the protein of interest has an N-terminal extension from the cleavage site and a another construct in which the protein of interest has no N-terminal extension when attached to the cleavage site.

FIG. 3 illustrates the structure of the cloning region used to attach a heterologous gene to the polynucleotide encoding the signal peptide such that the expressed heterologous protein has an extended N-terminal sequence that is attached to the signal peptidase cleavage site;

FIG. 4 illustrates the structure of the cloning region used to attach a heterologous gene to the polynucleotide encoding the signal peptide such that the expressed heterologous protein has no additional N-terminal sequences when attached to the signal peptidase cleavage site;

FIG. 5 illustrates the sequence of human Apolipoprotein and the starting amino acid residue of the Pre-pro Apo-A1, pro-Apo-A1, and Apo-A1.

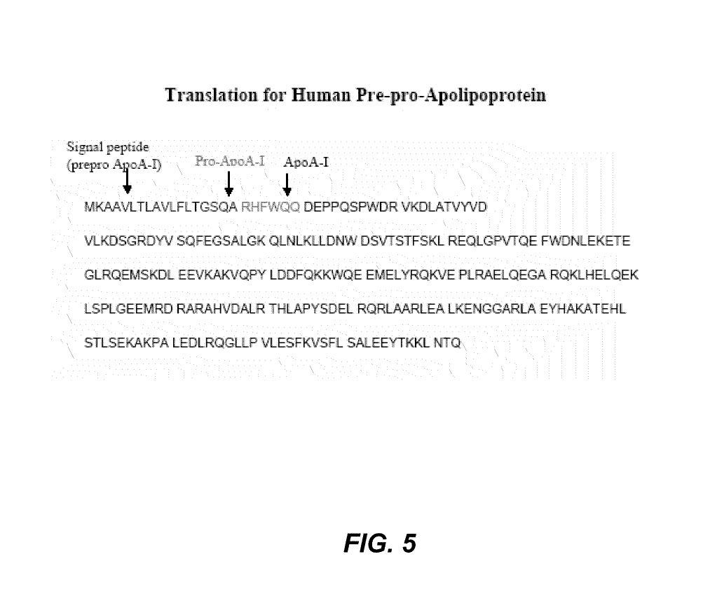

FIG. 6 illustrates the various fusion polypeptides designed for expression and secretion of Apo-A1 polypeptide;

FIG. 7 illustrates the P170 expression vectors used to express the Apo-A1 gene and polypeptide in L. lactis host cells; and

FIG. 8 illustrates the polynucleotide encoding Apo-A1 (SEQ ID NO:11) in which the codons have been optimized for expression in L. lactis spp cremori: the 5′ portion coding for amino acid residues S˜S˜A is removed upon cloning into the P170 based expression vector, such that the resulting Apo-A1 beginning with sequence D˜E˜P˜P is attached to the carboxy terminal alanine (A) residue of the signal peptide.

### 5. DETAILED DESCRIPTION

In high-throughput early discovery and high-yield production of candidate therapeutic proteins, E. coli based expression systems are widely used. However, not all proteins can be produced in high yields using E. coli as a host organism. In addition, successful recombinant protein expression/purification in E. coli depends on a high-fidelity system capable of rendering purified proteins free of contaminants, such as endotoxin. The prototypical examples of endotoxin are lipopolysaccharide (LPS) or lipo-oligo-saccharide (LOS) found in the outer membrane of various Gram-negative bacteria. In pharmaceutical preparations of therapeutic proteins, presence of such endotoxins must be minimized since even small amounts can have adverse consequences. The presence of endotoxin in purified protein samples obtained from E. coli is often undetected. Moreover, methods commonly used to remove contaminants, such as anion exchange chromatography, do not remove endotoxins (see, e.g., McKinstry, et al., 2003, Biotechniques 35:724-6).

Production of apolipoprotein A-I in E. coli is low (see, e.g., U.S. Pat. No. 5,059,528 and references cited therein; see also McGuire, et al., 1996, J Lipid Res. 37:1519-1528; Panagotopulos, et al., 2002, Protein Expr Purif. 25:353-61; and Ryan, et al., 2003, Protein Expr Purif. 27:98-103). Purification steps required to remove endotoxin can reduce the yield even more. Depending on the recombinant protein being expressed in E. coli, it may not be possible to eliminate the contaminating endotoxin and achieve a level of purity that complies with current Good Manufacturing Practice (cGMP) (see, e.g., Ma et al., 2004, Acta Biochim Biophys Sin. 36:419-24). For example, apolipoprotein A-I binds endotoxin (lipopolysaccharide (LPS)) and neutralizes its toxicity (Ma et al., supra). Thus, it may not be possible to develop an E. coli high-fidelity system capable of rendering purified recombinant apolipoproteins free of endotoxin. Production of apolipoproteins in other expression systems, such as yeast and insect cells, is also low (see, e.g., U.S. Pat. No. 5,059,528 and references cited therein).

In context of the above state of the art, the present disclosure provides compositions and methods for producing recombinant, secreted apolipoproteins in non-endotoxin producing bacteria, such as Gram-positive bacteria, including lactic acid bacteria. Some of the advantages of using non-endotoxin bacteria include, among others, (1) the absence of endotoxins, (2) the availability of lactic acid bacterial strains that do not produce extracellular proteases; (3) ease of manipulating lactic acid bacteria; (4) the ability of lactic acid bacteria to secrete recombinant peptides, polypeptides or proteins, which can be stable and easier to purify; (5) use of fermentative metabolism (e.g., fermentation occurring in the absence of oxygen) that simplifies the scaling up of protein production by reducing or eliminating the need for specially designed equipment needed for avoiding localized pockets of oxygen, which if present, can decrease cell growth and reduce yield; (6) the availability of inducible expression systems for increasing the yields of expressed gene products; and (7) long history of safe use of lactic acid bacteria in the food industry, making them attractive cloning hosts for the production of therapeutic proteins, such as apolipoproteins.

Many commercially significant proteins are produced by recombinant gene expression in appropriate prokaryotic or eukaryotic host cells. It is frequently desirable to isolate the expressed protein product after secretion into the culture medium or, in the case of gram-negative bacteria, into the “periplasmic space” or “periplasm,” between the inner and outer cell membranes. Secreted proteins are typically soluble and can be separated readily from contaminating host proteins and other cellular components. In many expression systems, the rate of secretion limits the overall yield of protein product, and a considerable amount of product accumulates as an insoluble fraction inside the cell from where it is difficult to isolate. There is therefore a need to provide improved methods for directing the secretion of heterologous proteins from bacteria and other host-cell types.

The entry of almost all secreted proteins to the secretory pathway, in both prokaryotes and eukaryotes, is directed by specific signal peptides at the N-terminus of the polypeptide chain which are cleaved off during secretion. The present disclosure provides recombinant nucleic acids encoding a defined set of signal peptides that efficiently directs secretion of apolipoproteins, which can then be isolated from the culture medium.

For the descriptions in this specification and the appended claims, the singular forms “a”, “an” and “the” include plural referents unless the context clearly indicates otherwise. Thus, for example, reference to “a protein” includes more than one protein, and reference to “a polynucleotide” refers to more than one polynucleotide. Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”

The section headings used herein are for organizational purposes only and not to be construed as limiting the subject matter described.

### 5.1 ABBREVIATIONS

The abbreviations used for the genetically encoded amino acids are conventional and are as follows:

| | | One-Letter |
|---|---|---|
| Amino Acid | Three-Letter | Abbreviation |
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When peptide sequences are presented as a string of one-letter or three-letter abbreviations (or mixtures thereof), the sequences are presented in the N→C direction in accordance with common convention.

The abbreviations used for the genetically encoding nucleosides are conventional and are as follows: adenosine (A); guanosine (G); cytidine (C); thymidine (T); and uridine (U). Unless specifically delineated, the abbreviated nucleotides may be either ribonucleosides or 2′-deoxyribonucleosides. The nucleosides may be specified as being either ribonucleosides or 2′-deoxyribonucleosides on an individual basis or on an aggregate basis. When specified on an individual basis, the one-letter abbreviation is preceded by either a “d” or an “r,” where “d” indicates the nucleoside is a 2′-deoxyribonucleoside and “r” indicates the nucleoside is a ribonucleoside. For example, “dA” designates 2′-deoxyriboadenosine and “rA” designates riboadenosine. When specified on an aggregate basis, the particular nucleic acid or polynucleotide is identified as being either an RNA molecule or a DNA molecule. Nucleotides are abbreviated by adding a “p” to represent each phosphate, as well as whether the phosphates are attached to the 3′-position or the 5′-position of the sugar. Thus, 5′-nucleotides are abbreviated as “pN” and 3′-nucleotides are abbreviated as “Np,” where “N” represents A, G, C, T or U. When nucleic acid sequences are presented as a string of one-letter abbreviations, the sequences are presented in the 5′→3′ direction in accordance with common convention, and the phosphates are not indicated.

### 5.2 DEFINITIONS

In the present disclosure, the technical and scientific terms used in the descriptions herein will have the meanings commonly understood by one of ordinary skill in the art, unless specifically defined otherwise. Accordingly, the following terms are intended to have the following meanings.

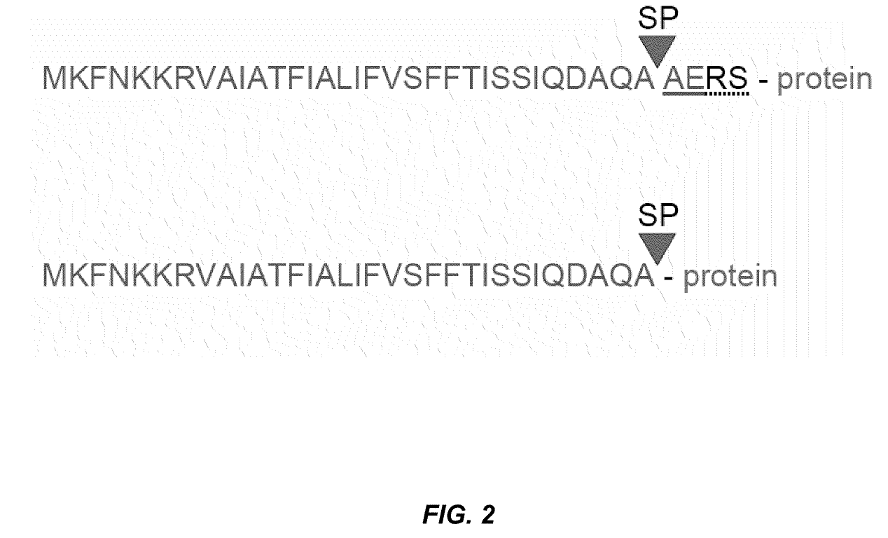

“Nucleobase” or “base” refers to those naturally occurring and synthetic heterocyclic moieties commonly known to those who utilize nucleic acid or polynucleotide technology or utilize polyamide or peptide nucleic acid technology to generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases illustrated in FIGS. 2(A) and 2(B) of Buchardt et al. (W0 92/20702 or W0 92/20703).

“Nucleoside” refers to a compound comprising a purine, deazapurine, or pyrimidine nucleobase, e.g., adenine, guanine, cytosine, uracil, thymine, 7-deazaadenine, 7-deazaguanosine, and the like, that is linked to a pentose at the 1′-position. When the nucleoside nucleobase is purine or 7-deazapurine, the pentose is attached to the nucleobase at the 9-position of the purine or deazapurine, and when the nucleobase is pyrimidine, the pentose is attached to the nucleobase at the 1-position of the pyrimidine, (see, e.g., Kornberg and Baker, 1992, DNA Replication, 2nd Ed., Freeman, San Francisco). The term “nucleotide” as used herein refers to a phosphate ester of a nucleoside, e.g., a triphosphate ester, wherein the most common site of esterification is the hydroxyl group attached to the C-5 position of the pentose. The term “nucleoside/tide” as used herein refers to a set of compounds including both nucleosides and nucleotides.

“Nucleobase polymer” or “Nucleobase oligomer” refers to two or more nucleobases that are connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, to several hundred nucleobases, to several thousand nucleobases, or more.

“Polynucleotides” or “Oligonucleotides” refers to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

“Protein,” “Polypeptide,” “Oligopeptide,” and “Peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

“Recombinant” when used with reference to, e.g., a cell, nucleic acid, polypeptide, expression cassette or vector, refers to a material, or a material corresponding to the natural or native form of the material, that has been modified by the introduction of a new moiety or alteration of an existing moiety using recombinant techniques, or is identical thereto but produced or derived from synthetic materials using recombinant techniques. For example, recombinant cells express genes that are not found within the native (non-recombinant) form of the cell (e.g., “exogenous nucleic acids”) or express native genes that are otherwise expressed at a different level, typically, under-expressed or not expressed at all.

“Recombinant host cell” refers to a cell that comprises a recombinant nucleic acid molecule. Thus, for example, recombinant host cells can express genes that are not found within the native (non-recombinant) form of the cell.

“Fusion construct” refers to a nucleic acid comprising the coding sequence for first polypeptide and the coding sequence (with or without introns) for a second polypeptide in which the coding sequences are adjacent and in the same reading frame such that, when the fusion construct is transcribed and translated in a host cell, a polypeptide is produced in which the C-terminus of the first polypeptide is joined to the N-terminus of the second polypeptide. A “fusion polypeptide” refers to the polypeptide product of the fusion construct.

“Fused,” “Joined” as used herein refers to linkage of heterologous amino acid or polynucleotide sequences. Thus, “fused” refers to any method known in the art for functionally connecting polypeptide and/or polynucleotide domains, including but not limited to recombinant fusion with or without intervening linking sequence, non-covalent association, and covalent bonding.

“Operably linked” refers to a functional relationship between two or more polynucleotide (e.g., DNA) segments. In some embodiments, it refers to the functional relationship of a transcriptional regulatory sequence to a transcribed sequence. For example, a promoter (defined below) is operably linked to a coding sequence, such as a nucleic acid, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

“Control sequence” refers to polynucleotide sequences used to effect the expression of coding and non-coding sequences to which they are associated. The nature of such control sequences differs depending upon the host organism. Control sequences generally include promoter, ribosomal binding site, and transcription termination sequence. The term “control sequences” is intended to include components whose presence can influence expression, and can also include additional components whose presence is advantageous, for example, leader sequences and fusion partner sequences.

“Promoter” refers to a nucleotide sequence capable of controlling the expression of a coding sequence or functional RNA. The promoter sequence can comprise proximal and more distal upstream elements, the latter elements often referred to as enhancers. Accordingly, an “enhancer” is a nucleotide sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. Promoter that can cause a nucleic acid fragment to be expressed in most cell types at most times are commonly referred to as a “constitutive promoter.” Promoters that can cause a nucleic acid fragment to be expressed in a regulatable matter are referred to as “inducible promoter.” It is further recognized that since in most cases the exact boundaries of regulatory sequences have not been completely defined, nucleic acid fragments of different lengths may have identical promoter activity

“Coding sequence” refers to that portion of a polynucleotide (e.g., a gene) that encodes an amino acid sequence of a polypeptide.

“Mature” protein refers to a post-translationally processed polypeptide; i.e., one from which any pre- or propeptides present in the primary translation product have been removed. “Precursor” protein refers to the primary product of translation of mRNA, i.e., with pre- and propeptides still present. Pre- and propeptides may be but are not limited to intracellular or extracellular localization signals.

“Vector” refers to a nucleic acid molecule capable of transporting another nucleic acid to which it has been linked. An example of a type of vector is an episome, e.g., a nucleic acid capable of extra-chromosomal replication. Vectors can be capable of autonomous replication and/or expression of nucleic acids to which they are linked. Vectors capable of directing the expression of genes to which they are operatively linked are referred to herein as “expression vectors.” In some embodiments, expression vectors of utility in recombinant DNA techniques are often in the form of “plasmids” which refer generally to circular double stranded DNA loops which, in their vector form are extrachromosomal (i.e., not part of the host chromosome).

“Secretion” refers to the process by which a protein is transported into the external cellular environment or, in the case of gram-negative bacteria, into the periplasmic space.

“Substitution” refers to the replacement of one or more nucleotides or amino acids by different nucleotides or amino acids, respectively, with respect to a reference sequence, such as, for example, a wild-type sequence.

“Insertion” or “addition” refers to a change in a nucleotide or amino acid sequence by the addition of one or more nucleotides or amino acid residues, respectively, as compared to a reference sequence, such as for example, a wild-type sequence.

“Deletion” refers to a change in the nucleotide or amino acid sequence by removal of one or more nucleotides or amino acid residues, respectively, from a reference sequence. For polypeptides, deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining biological activity of the reference polypeptide. Deletions can be directed to the internal portions and/or terminal portions of the nucleic acid or polypeptide. In various embodiments, the deletion can comprise a continuous segment or can be discontinuous.

“Hydrophobicity” refers to the distribution of apolar and polar residues along the length of a polypeptide sequence. Generally, hydrophobicity is expressed as a hydropathy scale based on the hydrophobic and hydrophilic properties of the 20 amino acids. A moving “window” of preset size determines the summed hydropathy at each point in the sequence (Y coordinate). These sums are then plotted against their respective positions (X coordinate). The window size can be varied, allowing the changes to the sensitivity of the calculation. Smaller windows result in “noisier” plots than do larger windows. Hydrophobicity scales and hydrophobicity calculations can use those known in the art. The Kyte-Doolittle scale is widely used for detecting hydrophobic regions in proteins. Regions with a positive value are hydrophobic. Short window sizes of 5-7 are generally used for predicting putative surface-exposed regions. Larger window sizes of 19-21 can be used for finding transmembrane domains if the values calculated are above 1.6 (Kyte and Doolittle, 1982, J Mol Biol, 157(1):105-132). Other hydrophobicity scales that can be used include, among others, Engelman et al., 1986, Annu Rev Biophys Biophys Chem 15:321-353; Sweet et al., 1983, J Mol Biol 171(4):479-488; Eisenberg et al., 1984, J Mol Biol, 179(1):125-142; Hopp et al., 1983, Mol Immunol 20(4):483-489; Cornette et al, 1987, J Mol Biol, 195(3):659-685; and Rose et al., 1985, Science, 229(4716):834-838; the disclosures of which are incorporated herein by reference. A “hydrophobic region” or “hydrophobic domain” of a polypeptide has on balance a higher degree of hydrophobic character than hydrophilic character. Under the Kyte-Doolittle system, hydrophobic regions have positive values in the hydropathy plot.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, Greene Publishing Associates, Inc. and John Wiley & Sons, Inc., (1995 Supplement)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc. Natl. Acad. Sci. USA 89:10915).

The degree of percent amino acid sequence identity can also be obtained by ClustalW analysis (version W 1.8) by counting the number of identical matches in the alignment and dividing such number of identical matches by the length of the reference sequence, and using the following default ClustalW parameters to achieve slow/accurate pairwise optimal alignments—Gap Open Penalty: 10; Gap Extension Penalty: 0.10; Protein weight matrix: Gonnet series; DNA weight matrix: IUB; Toggle Slow/Fast pairwise alignments=SLOW or FULL Alignment.

Exemplary determination of sequence alignment and % sequence identity can employ the BESTFIT or GAP programs in the GCG Wisconsin Software package (Accelrys, Madison Wis.), using default parameters provided, or the ClustalW multiple alignment program (available from the European Bioinformatics Institute, Cambridge, UK), using, in some embodiments, the parameters above.

“Reference sequence” refers to a defined sequence used as a basis for a sequence comparison, such as, for example, a wild-type sequence. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity.

“Comparison window” refers to a conceptual segment of at least about 20 contiguous nucleotide positions or amino acid residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Substantial identity” refers to a polynucleotide or polypeptide sequence that has at least 80 percent sequence identity, at least 85 percent sequence identity, about 90 to 95 percent sequence identity, and more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In some embodiments applied to polypeptides, the term “substantial identity” means that two polypeptide sequences, when optimally aligned, such as by the programs GAP or BESTFIT using default gap weights, share at least 80 percent sequence identity, preferably at least 90 percent sequence identity, at least 95 percent sequence identity, at least 98 percent sequence identity, at least 99 percent sequence identity, or more percent identity.

“Isolated polypeptide” refers to a polypeptide which is separated from other contaminants that naturally accompany it, e.g., other polypeptides, lipids, and polynucleotides. The term embraces polypeptides which have been removed or purified from their naturally-occurring environment or expression system (e.g., host cell or in vitro synthesis).

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure apolipoprotein composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

“Heterologous” when used with reference to a nucleic acid or polypeptide, refers to a sequence that comprises two or more subsequences which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences arranged in a manner not found in nature; e.g., a nucleic acid open reading encoding a protein of interest operatively linked to a promoter sequence inserted into an expression cassette, e.g., a vector.

“Position corresponding to” and “corresponding residue position” are used interchangeably to refer to a position of interest (i.e., base number or residue number) in a nucleic acid molecule or polypeptide relative to the position in another reference nucleic acid molecule or polypeptide. Generally, corresponding positions can be determined by comparing and aligning sequences to maximize the number of matching residues, for example, such that identity between the sequences is greater than 95%, greater than 96%, greater than 97%, greater than 98% and greater than 99%. The position of interest is then given the number assigned in the reference nucleic acid molecule or polypeptide. For example, a thirty eight amino acid reference sequence of residues X¹-X³⁸ is described herein for a signal peptide used to direct secretion of a protein of interest (e.g., apolipoprotein). To identify and describe another signal peptide, the sequences are aligned and then the position that lines up with the reference sequence is identified. Since the other signal peptide may be of different length or require the insertion of gaps for optimal alignment, a residue position on the other signal peptide may not be the identical position in the reference sequence, but instead is at a residue position “corresponding to” or “corresponding amino acid residue position” in the reference polypeptide sequence.

### 5.3 SIGNAL PEPTIDES FOR PRODUCING SECRETED APOLIPOPROTEIN

For producing secreted forms of apolipoprotein, the present disclosure provides a recombinant nucleic acid comprising a first polynucleotide encoding a signal peptide for directing secretion of an apolipoprotein encoded by a second polynucleotide, where the first and second polynucleotide are operably linked to direct secretion of the apolipoprotein. As used herein, the terms “signal sequence,” “signal peptide,” “leader peptide,” and “secretory leader” are used interchangeably and refer to a short, continuous stretch of amino acids generally positioned at the amino-terminus of polypeptides, which directs their delivery to various locations outside the cytosol (von Heijne et al., 1985, J. Mol. Biol. 184:99-105; Kaiser and Botstein, 1986, Mol. Cell. Biol. 6:2382-2391). In general, signal peptides usually comprise (i) an amino-terminal region that contains a number of positively charged amino acids, such as lysine and arginine; (ii) a central hydrophobic core of about 4-16 or more amino acids and; (iii) a hydrophilic carboxy-terminal region that contains a sequence motif recognized by a signal peptidase (von Heijne G, 1990, J. Membrane Biol. 115(3):195-201). Signal peptides in Gram positive bacteria vary from about 25 to over 36 amino acids in length (Martoglio and Dobberstein, 1998, Trends Cell Biol. 8(10):410-5).

In various embodiments, a first polynucleotide encoding the signal peptide can be operatively joined to a polynucleotide containing the coding region of the apolipoprotein in such manner that the signal peptide coding region is upstream of (e.g., 5′) and in the same reading frame with the apolipoprotein coding region to provide a fusion construct. The fusion construct can be expressed in a host cell to provide a fusion polypeptide comprising the signal peptide joined, at its carboxy terminus, to the recombinant polypeptide at its amino terminus. The fusion polypeptide can be secreted from the host cell. However, generally, the signal peptide is cleaved from the fusion polypeptide during the secretion process, resulting in the accumulation of secreted recombinant polypeptide in the external cellular environment or, in some cases, in the periplasmic space.

In some embodiments, the first polynucleotide sequence encodes a signal peptide comprising the structure:

(n)ₓ˜(m)_{y}˜(c)_{z},

wherein
each n is independently any amino acid residue, with two or more n being a basic amino acid residue.
each m is independently an aliphatic, aromatic, hydrophobic, or hydroxyl containing amino acid residue;
each c is independently any amino acid residue, with two or more c being a polar amino acid residue;
x is 6, 7, or 8;
y is any integer from 13 to 16;
z is any integer from 5 to 14; and
“˜” is a peptide bond.

In various embodiments, the recombinant nucleic acid encodes a polypeptide in which a signal peptide is attached at its carboxy terminus to the amino terminus of the expressed apolipoprotein. In some embodiments, y is 13, 14, or 16. In some embodiments, the structure of the signal peptide represented by (m)_{y} is hydrophobic. For example under the Kyte-Doolittle system, the (m)_{y} displays a positive value. In some embodiments, the (m)_{y} region has at least 7 hydrophobic, 9 hydrophobic, 10 hydrophobic, 12 hydrophobic, or up to all hydrophobic amino acids, as defined below. In some embodiments, the (m)_{y} region has in addition to the hydrophobic acids, one or more hydroxyl containing amino acid residues, up to three hydroxyl containing amino acid residues.

In some embodiments, the cleavage site recognized and acted on by the signal peptidase of a host organism is located in part of the signal peptide structure denoted by (c)_{z}. In some embodiments, the signal peptide can be cleaved by a signal peptidase in a Gram-positive bacterium, such as a lactic acid bacterium, used to express the recombinant polynucleotide.

In describing the amino acids that form the polypeptides herein, such as the signal peptide used to direct secretion of expressed apolipoprotein, the amino acid residues can be classified into various groups depending on the physical and chemical properties of the amino acid side chain. Accordingly, the following descriptions of the various classes of amino acids apply, unless specifically defined otherwise.

“Hydrophilic Amino Acid or Residue” refers to an amino acid or residue having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include L-Thr (T), L-Ser (S), L-His (H), L-Glu (E), L-Asn (N), L-Gln (O), L-Asp (D), L-Lys (K) and L-Arg (R).

“Acidic Amino Acid or Residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic Amino Acid or Residue” refers to a hydrophilic amino acid or residue having a side chain exhibiting a pK value of greater than about 6 when the amino acid is included in a peptide or polypeptide. Basic amino acids typically have positively charged side chains at physiological pH due to association with hydronium ion. Genetically encoded basic amino acids include L-His (H), L-Arg (R) and L-Lys (K).

“Polar Amino Acid or Residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (O), L-Ser (S) and L-Thr (T).

“Hydrophobic Amino Acid or Residue” refers to an amino acid or residue having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include L-Pro (P), L-Ile (I), L-Phe (F), L-Val (V), L-Leu (L), L-Trp (W), L-Met (M), L-Ala (A) and L-Tyr (Y).

“Aromatic Amino Acid or Residue” refers to a hydrophilic or hydrophobic amino acid or residue having a side chain that includes at least one aromatic or heteroaromatic ring. Genetically encoded aromatic amino acids include L-Phe (F), L-Tyr (Y) and L-Trp (W). Although owing to the pKa of its heteroaromatic nitrogen atom L-His (H) is classified above as a basic residue, as its side chain includes a heteroaromatic ring, it may also be classified as an aromatic residue.

“Non-polar Amino Acid or Residue” refers to a hydrophobic amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L-Leu (L), L-Val (V), L-Ile (I), L-Met (M) and L-Ala (A).

“Aliphatic Amino Acid or Residue” refers to a hydrophobic amino acid or residue having an aliphatic hydrocarbon side chain. Genetically encoded aliphatic amino acids include L-Ala (A), L-Val (V), L-Leu (L) and L-Ile (I).

The amino acid L-Cys (C) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges. The ability of L-Cys (C) (and other amino acids with —SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure L-Cys (C) is categorized as a polar hydrophilic amino acid, notwithstanding the general classifications defined above.

The amino acid Gly (G) is also unusual in that it bears no side chain on its α-carbon and, as a consequence, contributes only a peptide bond to a particular peptide sequence. Moreover, owing to the lack of a side chain, it is the only genetically-encoded amino acid having an achiral α-carbon. Although Gly (G) exhibits a hydrophobicity of 0.48 according to the normalized consensus scale of Eisenberg (Eisenberg et al., 1984, supra), for purposes of the present disclosure, Gly is categorized as an aliphatic amino acid or residue.

Owing in part to its conformationally constrained nature, the amino acid L-Pro (P) is also unusual. Although it is categorized herein as a hydrophobic amino acid or residue, it will typically occur in positions near the N- and/or C-termini so as not to deleteriously affect the structure of the compounds herein. However, as will be appreciated by skilled artisans, the compounds herein may include L-Pro (P) or other similar “conformationally constrained” residues at internal positions.

“Small Amino Acid or Residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include Gly, L-Ala (A), L-Val (V), L-Cys (C), L-Asn (N), L-Ser (S), L-Thr (T) and L-Asp (D).

“Hydroxyl-containing Amino Acid or Residue” refers to an amino acid or residue containing a hydroxyl (—OH) moiety. Genetically-encoded hydroxyl-containing amino acids include L-Ser (S) L-Thr (T) and L-Tyr (Y).

As will be appreciated by those of skill in the art, the above-defined categories are not mutually exclusive. For example, the delineated category of small amino acids includes amino acids from all of the other delineated categories except the aromatic category. Thus, amino acids having side chains exhibiting two or more physico-chemical properties can be included in multiple categories. As a specific example, amino acid side chains having heteroaromatic moieties that include ionizable heteroatoms, such as His, may exhibit both aromatic properties and basic properties, and can therefore be included in both the aromatic and basic categories. The appropriate classification of any amino acid or residue will be apparent to those of skill in the art, especially in light of the detailed disclosure provided herein.

In some embodiments, the signal peptide encoded by the recombinant nucleic acid comprises a polypeptide of residues X¹ to X³⁸, where X represents the amino acid and the superscript represents the residue position. In reference to the signal peptide above, in some embodiments, the (n)ₓ structure comprises the amino acid sequence
X¹˜X²˜X³˜X⁴˜X⁵˜X⁶˜X⁷˜X⁸,

wherein
X¹ is M;
X² is a basic amino acid;
X³ is an aromatic amino acid;
X⁴ is a basic or polar amino acid;
X⁵ is a basic amino acid;
X⁶ is a basic amino acid;
X⁷ is a basic amino acid;
X⁸ is an aliphatic amino acid; and

wherein
optionally each of X³ and X⁴ are independently absent.

In some embodiments, the structure (m)_{y} of the signal peptide comprises the amino acid sequence:

X⁹˜X¹⁰˜X¹¹˜X¹²˜X¹³˜X¹⁴˜X¹⁵˜X¹⁶˜X¹⁷˜X¹⁸˜X¹⁹˜X²⁰˜X²¹˜X²²˜X²³˜X²⁴,

wherein
X⁹ is an aliphatic amino acid; and
X¹⁰ is an aliphatic amino acid;
X¹¹ is an aliphatic amino acid:
X¹² is an aliphatic or hydroxyl containing amino acid;
X¹³ is an aromatic, aliphatic, or hydrophobic amino acid;
X¹⁴ is an aliphatic amino acid;
X¹⁵ is an aliphatic, aromatic, or hydrophobic amino acid;
X¹⁶ is an aliphatic amino acid;
X¹⁷ is an aliphatic amino acid;
X¹⁸ is an aliphatic, aromatic amino, or hydrophobic amino acid;
X¹⁹ is an aliphatic amino acid;
X²⁰ is an aliphatic, aromatic, hydrophobic or a hydroxyl containing amino acid;
X²¹ is an aliphatic, aromatic, or hydrophobic amino acid;
X²² is an aliphatic, aromatic, or hydrophobic amino acid;
X²³ is a aliphatic or hydroxyl containing amino acid; and
X²⁴ is an aliphatic amino acid.

In some embodiments, the structure (c)_{z} of the signal peptide comprises the amino acid sequence
X²⁵˜X²⁶˜X²⁷˜X²⁸˜X²⁹˜X³⁰˜X³¹˜X³²˜X³³˜X³⁴˜X³⁵˜X³⁶˜X³⁷˜X³⁸,

wherein
X²⁵ is a hydroxyl containing amino acid:
X²⁶ is a hydroxyl containing amino acid;
X²⁷ is an aliphatic amino acid;
X²⁸ is a polar or constrained amino acid;
X²⁹ is an acidic amino acid;
X³⁰ is a polar or aliphatic amino acid;
X³¹ is a polar or hydroxyl containing amino acid;
X³² is an aliphatic or hydroxyl containing amino acid;
X³³ is an polar amino acid;
X³⁴ is an aliphatic amino acid;
X³⁵ is an aliphatic or acidic amino acid;
X³⁶ is an acidic or hydroxyl containing amino acid;
X³⁷ is a basic amino acid; and
X³⁸ is a hydroxyl containing amino acid; and

wherein
optionally each of X²⁵, X²⁶, X²⁸, X²⁹, X³², X³³, X³⁴, X³⁵, X³⁶, X³⁷ and X³⁸ is independently absent.

In some embodiments where amino residue are absent in the (c)_{z} part of the signal peptide, the number of amino acid residue absent can be 1 or more, 2 or more, 3 or more, 5 or more, up to 9 amino acid residues, although more can be absent as long as a functional signal peptide is preserved. In some embodiments, one or more of amino acid residues X³³, X³⁴, X³⁵, X³⁶, X³⁷, and X³⁸ are absent. In some embodiments, all of amino acid residues X³³, X³⁴, X³⁵, X³⁶, X³⁷, and X³⁸ are absent.

In some embodiments, the recombinant nucleic acid encodes a signal peptide which has homology to the signal peptide of any one of specified polypeptides selected from SEQ ID NOS:1-10. In some embodiments, the encoded signal peptide has at least 60%, at least 70%, at least 80%, at least 85%, at least 90%, at least 95%, at least 96%, at least 97%, at least 98%, or at least 99% or more sequence identity as compared to a reference sequence selected from the group consisting of SEQ ID NOS:1-10.

In some embodiments, the recombinant nucleic acid encodes a signal peptide which has homology to the signal peptide of SEQ ID NO: 1. In some embodiments, the signal peptides can have at least 60% or more sequence identity, 70% or more sequence identity, 80% or more sequence identity, 85% or more sequence identity, 90% or more sequence identity, 95% or more sequence identity, 96% or more sequence identity, 97% or more sequence identity, 98% or more sequence identify, or 99% or more sequence identity as compared to the signal peptide of SEQ ID NO: 1.

In some embodiments, the recombinant nucleic acid encodes a signal peptide which has homology to the signal peptide of SEQ ID NO:2. In some embodiments, the signal peptides can have at least 60% or more sequence identity, 70% or more sequence identity, 80% or more sequence identity, 85% or more sequence identity, 90% or more sequence identity, 95% or more sequence identity, 96% or more sequence identity, 97% or more sequence identity, 98% or more sequence identify, or 99% or more sequence identity a compared to the signal peptide of SEQ ID NO:2.

In some embodiments, the recombinant nucleic acid encodes a signal peptide which has homology to the signal peptide of SEQ ID NO:10. In various embodiments, the signal peptides can have at least 60% or more sequence identity, 70% or more sequence identity, 80% or more sequence identity, 85% or more sequence identity, 90% or more sequence identity, 95% or more sequence identity, 96% or more sequence identity, 97% or more sequence identity, 98% or more sequence identify, or 99% or more sequence identity a compared to the signal peptide of SEQ ID NO:10.

In some embodiments, the recombinant nucleic acid encodes a signal peptide selected from the group consisting of SEQ ID NO: 1, 2, 3, 4, 5, 6, 7, 8, 9, and 10. Other signal peptides sequences useful for the purposes herein are described in Ravn et al., 2003, Microbiology 149, 2193-2201 and US 2004/0038263, the disclosures of which are incorporated herein by reference.

In various embodiments, the signal peptides useful for directing secretion of the apolipoprotein encoded by the second polynucleotide can comprise modifications to the signal peptides above. Modifications can comprise substitutions, insertions, and/or deletions of the amino acid residues of a reference signal peptide sequence.

In reference to substitutions, the replacement amino acid can be a non-conservative or conservative substitution. “Non-conservative amino acid substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Conservative amino acid substitution” refers to the interchangeability of residues having similar side chains, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, isoleucine, and methionine; an amino acid with hydroxyl side chain is substituted with another amino acid with a hydroxyl side chain, e.g., serine and threonine; an amino acids having aromatic side chains is substituted with another amino acid having an aromatic side chain, e.g., phenylalanine, tyrosine, tryptophan, and histidine; an amino acid with a basic side chain is substituted with another amino acid with a basis side chain, e.g., lysine, arginine, and histidine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

In various embodiments herein, the substitutions for generating signal peptide sequences can comprise conservative substitutions, non-conservative substitutions, as well as combinations of conservative and non-conservative substitutions.

In some embodiments, the encoded signal sequence comprises one or more amino acid substitutions or deletions of SEQ ID NO:1 at corresponding amino acid residue positions selected from: X³, X⁴, X⁸, X⁹, X¹⁰; X¹¹, X¹², X¹³, X¹⁴, X¹⁵, X¹⁷, X¹⁸, X¹⁹, X²⁰, X²¹, X²², X²³, X²⁵, X²⁶, X²⁸, X²⁹, X³⁰, X³¹, X³², X³³, X³⁵, X³⁶, X³⁷, and X³⁸,

In some embodiments, the amino acid substitutions are selected from the following:

X³ is an aromatic amino acid other than F;

X⁴ is a basic amino acid or polar amino acid other than N;

X⁸ is an aliphatic amino acid other than V;

X⁹ is an aliphatic amino acid other than A;

X¹⁰ is an aliphatic amino acid other than I;

X¹¹ is an aliphatic amino acid other than I

X¹² is an aliphatic amino acid or S;

X¹³ is an aliphatic amino acid or a hydrophobic or aromatic amino acid other than F;

X¹⁴ is an aliphatic amino acid other than I;

X¹⁵ is an aromatic amino acid or a hydrophobic or aliphatic amino acid other than A;

X¹⁷ is an aliphatic amino acid other than I;

X¹⁸ is an aliphatic amino acid or hydrophobic or aromatic amino acid other than F;

X¹⁹ is an aliphatic amino acid other than V;

X²⁰ is an aliphatic, aromatic, or hydrophobic amino acid, or T;

X²¹ is an aliphatic amino acid or a hydrophobic or aromatic amino acid other than F;

X²² is an aliphatic amino acid or a hydrophobic or aromatic amino acid other than F;

X²³ is an aliphatic amino acid or S;

X²⁸ is a constrained amino acid or a polar amino acid other than Q;

X³⁰ is an aliphatic amino acid or polar amino acid other than N;

X³¹ is an hydroxyl containing amino acid or polar amino acid other than Q;

X³² is a hydroxyl containing amino acid or an aliphatic amino acid other than A;

X³³ is a polar amino acid other than N;

X³⁵ is an acidic amino acid or an aliphatic amino acid other than A; and

X³⁶ is a hydroxyl containing amino acid residue or a D.

In some embodiments, the encoded signal peptide sequence comprises up to 14 non-conservative substitutions at amino acid residue positions selected from X⁴, X¹², X¹³, X¹⁵, X¹⁸, X²⁰, X²¹, X²², X²³, X²⁸, X³⁰, X³¹, X³², X³⁵, and X³⁶ of SEQ ID NO:1, and where applicable the corresponding amino acid residue positions of SEQ ID NO:2, and optionally one or more conservative substitutions at other amino acid residue positions.

In some embodiments, non-conservative amino acid substitutions are selected from the following:

X⁴ is a basic amino acid;

X¹² is an aliphatic amino acid;

X¹³ is an aliphatic amino acid;

X¹⁵ is an aromatic amino acid;

X¹⁸ is an aliphatic amino acid;

X²⁰ is an aliphatic, aromatic, or hydrophobic amino acid;

X²¹ is an aliphatic amino acid;

X²² is an aliphatic amino acid;

X²³ is an aliphatic amino acid;

X²⁸ is a constrained amino acid;

X³⁰ is an aliphatic amino acid;

X³¹ is an hydroxyl containing amino acid;

X³² is a hydroxyl containing amino acid;

X³⁵ is an acidic amino acid; and

X³⁶ is a hydroxyl containing amino acid.

In some embodiments above, the amino acid residues of the signal peptide are optionally absent (e.g., deleted) at one or more corresponding amino acid residue positions of selected from:
X³, X⁴, X²⁵, X²⁶, X²⁸, X²⁹, X³², X³³, X³⁵, X³⁶, X³⁷, and X³⁸.

In some embodiments, the amino acid residues absent are selected from the corresponding amino acid residue positions X³³, X³⁴, X³⁵, X³⁶, X³⁷, and X³⁸ of SEQ ID NO:1 reference sequence. In certain embodiments, all amino acid residues at corresponding positions X³³, X³⁴, X³⁵, X³⁶, X³⁷, and X³⁸ are absent.

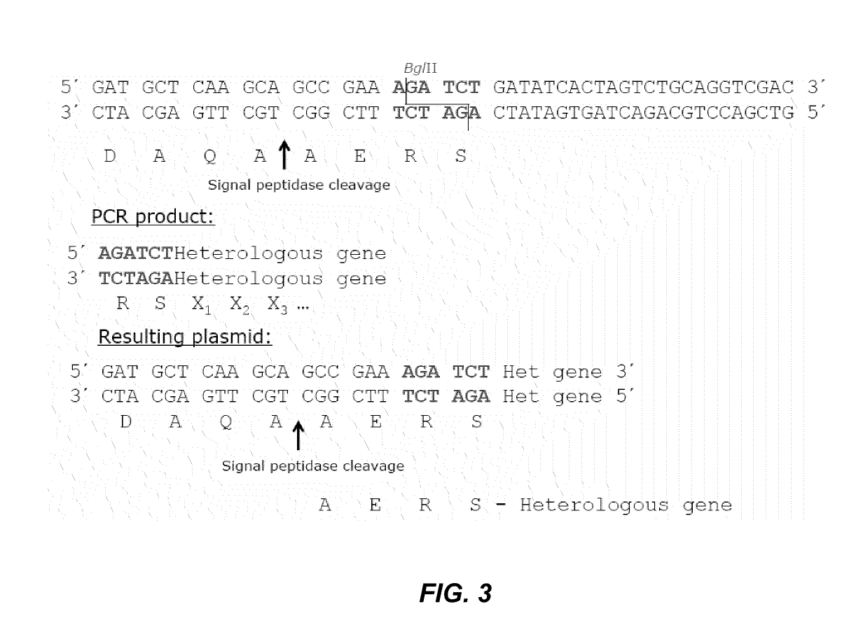

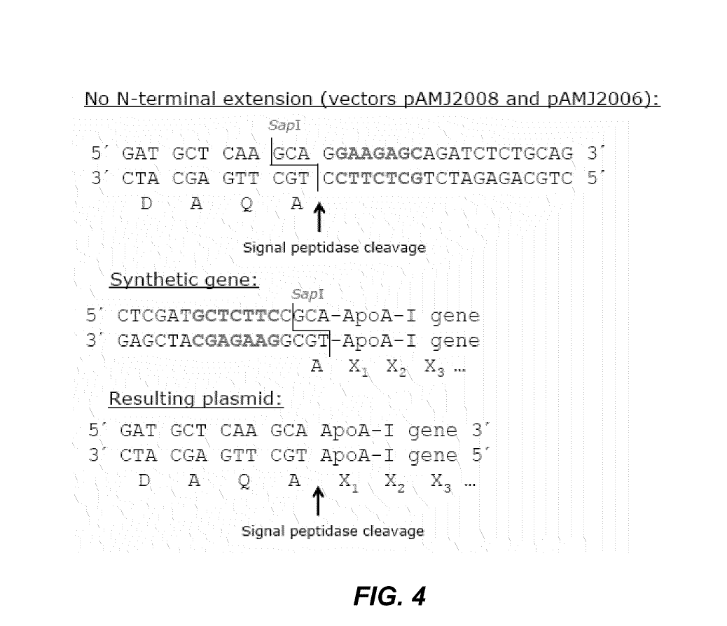

As noted above, the signal peptide encoded by the first polynucleotide can have a cleavage site for a signal peptidase. Accordingly, in some embodiments, the signal peptide can terminate at a signal peptidase cleavage site. Various signal peptidase cleavage sites have been described for Gram positive bacteria (see, e.g., Sibakov et al., 1991, Applied Environ Microbiol. 57(2):341-348; Pragai et al., 1997, Microbiology 143:1327-1333; Bolhuis et al., 1999, J Biol. Chem. 274(25):24585-24592; Tjalsma et al., 1997, J Biol. Chem. 272(41):25983-25992; and van Roosmalen et al., 2004, Biochim Biophys Acta 1694(1-3):279-97; all publications incorporated herein by reference), and thus may be applied to the signal peptides described here. In some embodiments, the signal peptidase cleavage site are the cleavage sites presented in SEQ ID NOS: 1-10 and those presented in FIGS. 2-4. In some embodiments, the signal peptidase cleavage site is between amino acid residues corresponding to residues X³² and X³³ of SEQ ID NO:1. In some embodiments, the signal peptide terminates at amino acid residue corresponding to residue X³², which can be an alanine (A) in some specific embodiments described herein.

In the various embodiments described herein, the encoded signal peptide is a “functional” signal peptide. The term “functional” refers to a polypeptide which possesses either the native biological activity of the naturally-produced polypeptide of its type, or any specific desired activity, which for a signal peptide is directing secretion of the apolipoprotein encoded by the second polynucleotide. In some embodiments, the function signal peptide can be cleaved by a host cell signal peptidase, such as a signal peptidase in a lactic acid bacterium.

As will be apparent to the skilled artisan, the ability of the signal peptides to direct secretion of the protein of interest can be determined using well known techniques. There are various assays known to those of skill in the art for detecting and measuring activity of secreted polypeptides. In some embodiments, polynucleotides encoding reporter molecules can be used. Exemplary reporter molecules include, among others, green fluorescent protein (GFP), β-galactosidase, horseradish peroxidase, proteases and glucouronidase. In particular, for proteases, the assays can be based on the release of acid-soluble peptides from casein or hemoglobin measured as absorbance at 280 nm or calorimetrically using the Folin method (see, e.g., Bergmeyer, et al., “Methods of Enzymatic Analysis,” in Peptidases, Proteinases and their Inhibitors, Vol 5, Verlag Chemie, Weinheim (1984)). Other assays can involve the solubilization of chromogenic substrates (Ward, “Proteinases,” in Microbial Enzymes and Biotechnology, (W. M. Fogarty, ed.) Applied Science, London, pg. 251-317 (1983)).

Among the reporters that can be used in L. lactis and other lactic acid bacterial species, genes coding for nucleases, including the Staphylococcus aureus nuclease (Nuc), has been shown to be useful as secretion reporter (Poquet et al., supra). Nuc is suitable for the examining signal peptide activity since the protein is inactive intracellularly and its structure is a simple a monomer lacking disulfide bonds. Furthermore, the codon usage in the nuc gene is suitable for high level expression in lactococci, and the plate assay for detection of secretion is not toxic, eliminating the need for replica plating. Following testing with the reporter molecule, the signal peptides can be tested for directing secretion of the apolipoprotein.

In some embodiments, the methods for determining the secreted levels of a heterologous polypeptide in a host cell include use of polyclonal or monoclonal antibodies specific for the polypeptide (e.g., apolipoprotein itself or epitope tags on the apolipoprotein). Examples include Western blotting, enzyme-linked immunosorbent assay (ELISA), radioimmunoassay (RIA), and fluorescent activated cell sorting (FACS). These and other assays are described in, among others, Hampton et al, Serological Methods: A Laboratory Manual, APS Press, St Paul, Minn. (1990) and Maddox et al, 1983, J Exp Med 158:1211; the disclosures of which are incorporated herein by reference.

Other techniques for detecting the secreted polypeptide that can be used alone or in various combinations, include, among others, gel electrophoresis, isoelectrofocusing, mass spectrometry, and chromatography (e.g., high pressure or high performance liquid chromatography). All of these techniques are well known to the skilled artisan.

### 5.4 APOLIPOPROTEINS, APOLIPOPROTEIN PEPTIDES, AND CORRESPONDING POLYNUCLEOTIDES

In various embodiments herein, the first polynucleotide encoding the signal peptide is operably linked to a second polynucleotide encoding an apolipoprotein to direct secretion of the expressed apolipoprotein. The nature of the apolipoproteins expressed recombinantly in a host cell is not critical for success. Virtually any apolipoprotein and/or derivative or analog thereof that provides therapeutic and/or prophylactic benefit as described herein can be expressed in one of more of the members comprising the Gram-positive bacteria, such as a lactic acid bacteria. Moreover, any alpha-helical peptide or peptide analog, or any other type of molecule that “mimics” the activity of an apolipoprotein (e.g., ApoA-I) in that it can activate LCAT or form discoidal particles when associated with lipids, can be expressed recombinantly in Gram-positive bacterium, and is therefore included within the definition of “apolipoprotein.” Examples of suitable apolipoproteins include, but are not limited to, preproapolipoprotein forms of ApoA-I, ApoA-II, ApoA-IV, ApoA-V, and ApoE; pro- and mature forms of human ApoA-I, ApoA-II, ApoA-IV, and ApoE; and active polymorphic forms, isoforms, variants and mutants as well as truncated forms, the most common of which are ApoA-IM (ApoA-IM) and ApoA-IP (ApoA-IP). ApoA-IM is the R173c molecular variant of ApoA-I (see, e.g., Parolini et al., 2003, J Biol. Chem. 278(7):4740-6; Calabresi et al., 1999, Biochemistry 38:16307-14; and Calabresi et al., 1997, Biochemistry 36:12428-33). ApoA-IP is the R151 molecular variant of ApoA-I (see, e.g., Daum et al., 1999, J Mol. Med. 77(8):614-22). Apolipoproteins mutants containing cysteine residues are also known, and can also be used (see, e.g., U.S. Publication 2003/0181372). The apolipoproteins may be in the form of monomers or dimers, which may be homodimers or heterodimers. For example, homo- and heterodimers of pro- and mature apolipoproteins that can be prepared include, among others, apoliApoA-I (Duverger et al., 1996, Arterioscler Thromb Vasc Biol. 16(12):1424-29), ApoA-IM (Franceschini et al., 1985, J Biol. Chem. 260:1632-35), ApoA-IP (Daum et al., 1999, J Mol. Med. 77:614-22), ApoA-I (Shelness et al., 1985, J Biol. Chem. 260(14):8637-46; Shelness et al., 1984, J Biol. Chem. 259(15):9929-35), ApoA-IV (Duverger et al., 1991, Euro J. Biochem. 201(2):373-83), ApoE (McLean et al., 1983, J Biol. Chem. 258(14):8993-9000), ApoJ and ApoH. The apolipoproteins may include residues corresponding to elements that facilitate their isolation, such as His tags or antibody tags, or other elements designed for other purposes, so long as the apolipoprotein retains some functional activity when included in a complex.

In some embodiments, the nucleotide sequences encoding the apolipoproteins are obtained from humans. Non-limiting examples of human apolipoprotein sequences are disclosed in U.S. Pat. Nos. 5,876,968; 5,643,757; and 5,990,081, and WO 96/37608; the disclosures of which are incorporated herein by reference in their entireties.

In addition to the references above, sequences for human apolipoproteins include sequences available in various sequence databases, such as Genbank. For instance, Genbank Accession Nos. for human ApoA-1 include, but are not limited to, NP_{—}000030 and AAB59514, PO2647, CAA30377, and AAA51746. GenBank Accession No. for human ApoA-II include, but are not limited to NP_{—}001634 and PO2652. GenBank Accession Nos. for human ApoA-IV include, but are not limited to, AAB50137, PO6727, NP_{—}000473, and NP_{—}001634. GenBank Accession Nos. for human ApoA-V include, but are not limited to, NP_{—}443200, AAB59546, and Q6Q788. GenBank Accession Nos. for human ApoE include, but are not limited to, Q6Q788, PO2649, AAB50137, BAA96080, AAG27089, AAL82810, AAB59546, AAB59397, AAH03557, AAD02505, NP_{—}000032, and AAB59518.

In some embodiments, the nucleotide sequences encoding the apolipoproteins are obtained from non-humans (see, e.g., U.S. Publication 2004/0077541, the disclosure of which is incorporated herein by reference). Apolipoprotein A-I protein has been identified in a number of non-human animals, for example, cows, horses, sheep, monkeys, baboons, goats, rabbits, dogs, hedgehogs, badgers, mice, rats, cats, guinea pigs, hamsters, duck, chicken, salmon and eel (Brouillette et al., 2001, Biochim Biophys Acta. 1531:4-46; Yu et al., 1991, Cell Struct Funct. 16(4):347-55; Chen and Albers, 1983, Biochim Biophys Acta. 753(1):40-6; Luo et al., 1989, J Lipid Res. 30(11):1735-46; Blaton et al., 1977, Biochemistry 16:2157-63; Sparrow et al., 1995, J Lipid Res. 36(3):485-95; Beaubatie et al., 1986, J Lipid Res. 27:140-49; Januzzi et al., 1992, Genomics 14(4):1081-8; Goulinet and Chapman, 1993, J Lipid Res. 34(6):943-59; Collet et al., 1997, J Lipid Res. 38(4):634-44; and Frank and Marcel, 2000, J Lipid Res. 41(6):853-72).

Apolipoprotein A-I protein derived from non-human animal species are of similar size (Mr≈27,000-28,000) and share considerable homology (Smith et al., 1978, Ann Rev Biochem. 47:751-7). For example, bovine ApoA-I protein comprises 241 amino acid residues and can form a series of repeating amphipathic alpha-helical regions. There are 10 amphipathic alpha-helical regions in bovine ApoA-I protein, typically occurring between residues 43-64, 65-86, 87-97, 98-119, 120-141, 142-163, 164-184, 185-206, 207-217 and 218-241 (see, e.g., Sparrow et al., 1992, Biochim Biophys Acta. 1123:145-150; and Swaney, 1980, Biochim Biophys Acta. 617:489-502). An amino acid sequence comparison between human ApoA-1 protein (GenBank Accession Nos. XM_{—}52106 or NM_{—}000039) and bovine ApoA-I protein (GenBank Accession No. A56858) using the program BLAST reveals that the sequences are 77% identical (Altschul et al., 1990, J Mol. Biol. 215(3):403-10).

Pig (porcine) ApoA-I protein comprises about 264 amino acid residues with a molecular weight of about 30,280. GenBank Accession No. S31394, provides a 264 residue porcine ApoA-I sequence with a molecular weight 30,254, while GenBank Accession No. JT0672 provides a 265 residue porcine ApoA-I protein with a molecular weight of 30,320 (see also, Weiler-Guttler et al., 1990, J. Neurochem. 54(2):444-450; Trieu et al., 1993, Gene 123(2):173-79; and Trieu et al., 1993, Gene 134(2):267-70).

Chicken ApoA-I precursor has 264 amino acid residues; the sequence of which is provided at GenBank Accession No. LPCHA1. Jackson et al., have described When ApoA-I as comprising 234 amino acid residues, having a molecular weight of about 28,000 and differing from human ApoA-I by the presence of isoleucine (Jackson et al., 1976, Biochim Biophys Acta. 420(2):342-9). Yang et al., describes mature chicken ApoA-I protein as being comprised of 240 amino acid residues with a less than 50% homology with humans (Yang et al., 1987, FEBS Lett. 224(2):261-6; see also, Shackelford and Lebherz, 1983, J Biol. Chem. 258(11):7175-7180; Banjeijee et al., 1985, J. Cell Biol. 101(4):1219-1226; Rajavashisth et al., 1987; J Biol. Chem. 262(15):7058-65; Ferrari et al., 1987, Gene 60(1):39-46; Bhattacharyya et al., 1991, Gene 104(2):163-168; and Lamon-Fava et al., 1992, J Lipid Res. 33(6):831-42). Circular dichroism studies of chicken ApoA-I protein demonstrate that the protein organizes as a bundle of amphipathic alpha-helices in a lipid free state (Kiss et al., 1999, Biochemistry 38(14):4327-34). A comparison of secondary structural features among chicken, human, rabbit, dog and rat indicates good conservation of ApoA-I secondary structure with human ApoA-I, especially in the N-terminal two-thirds of the protein (Yang et al., supra).

Lipoprotein studies in turkeys have identified an ApoA class of lipoprotein designated in analogy to human ApoA-I and ApoA-II. ApoA-I in turkeys is the major ApoA polypeptide with a molecular weight of about 27,000 (Kelley and Alaupovic, 1976, Atherosclerosis 24(1-2):155-75; and Kelley and Alaupovic, 1976, Atherosclerosis 24(1-2):177-87). Duck ApoA-I comprises about 246 amino acid residues and has a molecular weight of about 28,744 (GenBank Accession No. A61448; Gu et al., 1993, J Protein Chem. 12(5):585-91).

Non-limiting examples of peptides and peptide analogs that correspond to apolipoproteins, as well as agonists that mimic the activity of ApoA-I, ApoA-IM, ApoA-II, ApoA-IV, and ApoE, that are suitable for expression in lactic acid bacteria are disclosed in U.S. Pat. Nos. 6,004,925; 6,037,323; 6,046,166; and 5,840,688; U.S. publications 2004/0266671, 2004/0254120, 2003/0171277, 2003/0045460, and 2003/0087819, the disclosures of which are incorporated herein by reference in their entireties).

As will be apparent to the skilled artisan, because of the knowledge of the codons corresponding to the various amino acids, availability of a polypeptide sequence provides a description of all polynucleotides capable of encoding the subject polypeptides. The degeneracy of the genetic code, where the same amino acids are encoded by alternative or synonymous codons allows an extremely large number of polynucleotides to be made, all of which encode the fusion polypeptides described herein. Thus, having identified a particular amino acid sequence, those skilled in the art can make any number of different nucleic acids by simply modifying the sequence of one or more codons in a way which does not change the amino acid sequence of the protein. Consequently, the present disclosure specifically contemplates each and every possible variation of polynucleotides that could be made by selecting combinations based on the possible codon choices, and all such variations are to be considered specifically disclosed for any polypeptide disclosed herein.

In some embodiments, the recombinant polynucleotides encoding the apolipoprotein, and/or the signal peptide, are codon optimized for expression in a particular host cell. “Codon optimized” refers to changes in the codons of the polynucleotide encoding a polypeptide to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome.

The terms “preferred,” “optimal,” or “high codon usage bias” codons refer interchangeably to codons that are used at higher frequency in the protein coding regions as compared to other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression.

A variety of methods are known for determining the codon frequency (e.g., codon usage, relative synonymous codon usage) and codon preference in specific organisms, including multivariat analysis, for example, using cluster analysis or correspondence analysis, and the effective number of codons used in a gene (see, e.g., GCG CodonPreference, Genetics Computer Group Wisconsin Package; CodonW, John Peden, University of Nottingham; McInerney, J. O, 1998, Bioinformatics 14:372-73; Stenico et al., 1994, Nucleic Acids Res. 222437-46; and Wright, F., 1990, Gene 87:23-29). An exemplary method for codon optimizing the coding sequence is the GeneOptimizer® sequence optimization software (Geneart, Inc., Toronto, Calif.), as described in WO2004059556 and WO2006015789, which are incorporated herein by reference.

Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

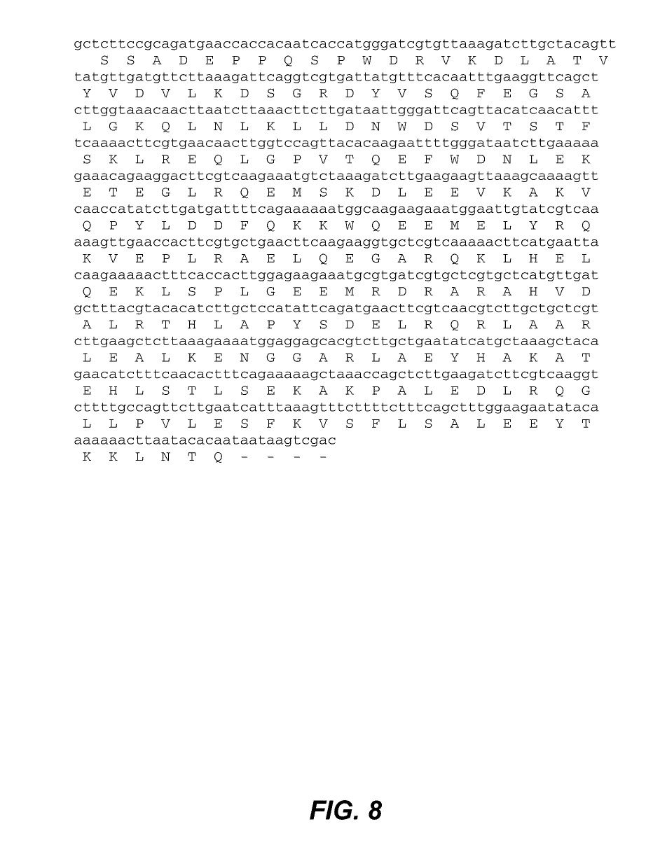

In various embodiments, the codons are preferably selected to fit the host cell in which the polypeptide is being produced. For example, preferred codons used in bacteria are used to express the gene in bacteria; preferred codons used in yeast are used for expression in yeast; and preferred codons used in mammals are used for expression in mammalian cells. For example, the codons selected for the polynucleotide of FIG. 8 (SEQ ID NO:11) is for the host cell Lactobacillus lactis spp cremoris. Other Gram-positive bacteria, such as lactic acid bacterial host cells for codon optimization, as further described below, include, but are not limited to, Lactococcus spp., Streptococcus spp., Lactobacillus spp., Leuconostoc spp., Pediococcus spp., Brevibacterium spp. and Propionibacterium spp.

In some embodiments, all codons need not be replaced to optimize the codon usage since the natural sequence will comprise preferred codons and because use of preferred codons may not be required for all amino acid residues. Consequently, codon optimized polynucleotides encoding the signal peptide and/or apolipoprotein may contain preferred codons at about 40%, 50%, 60%, 70%, 80%, or greater than 90% of codon positions of the full length coding region.

### 5.5 EXPRESSION VECTORS AND CONTROL SEQUENCES

In various embodiments, the recombinant polynucleotide encoding the signal peptide and apolipoprotein can comprise part of an expression vector which has at least one control sequence. The term “control sequence” is defined herein to include all components, which are necessary or advantageous for the expression of a polypeptide of interest. Each control sequence may be native or foreign to the nucleic acid sequence encoding the polypeptide. Control sequences include, but are not limited to, promoters, ribosome binding sites, and transcription terminator. In some embodiments, the control sequences include a promoter, ribosome binding site, and transcriptional and translational stop signals. The control sequences may be provided with linkers for the purpose of introducing specific restriction sites facilitating ligation of the control sequences with the coding region of the nucleic acid sequence encoding a polypeptide.

In some embodiments, the expression vector comprises at least one promoter, such as a constitutive or regulatable promoter, operably linked to the coding polynucleotide sequence. The promoter is operably linked such that the regulatory element is in the appropriate location and orientation in relation to a polynucleotide coding sequence to control RNA polymerase initiation and expression of the polynucleotide. In some embodiments, the promoter region can be based on a promoter present in any prokaryotic cell, and which promoter is capable of functioning in a Gram-positive bacteria (e.g., promoting expression of an operably linked nucleic acid sequence). Exemplary promoters include, among others, beta-lactamase (penicillinase) and lactose (lac) promoter systems, the tryptophan (trp) promoter system, and the arabinose promoter system. It is to be understood that any available promoter system compatible with prokaryotes can be used (see, e.g., Baneyx, F., 1999, Curr. Opinion Biotech. 10:411-421, and U.S. Pat. No. 5,698,435).

In some embodiments, the promoter is derived from a Gram-positive bacterial species. For example, the promoter region can be derived from a promoter region of Lactococcus lactis including Lactococcus lactis subspecies lactis, e.g. the strain designated MG1363 (also referred to in the literature as Lactococcus lactis subspecies cremoris) (Nauta et al., 1997, Nat. Biotechnol. 15:980-983), and Lactococcus lactis subspecies lactis biovar. diacetylactis. Exemplary promoters are described in Israelsen et al., 1995, Appl. Environ. Microbiol. 61(7):2540-2547; den Hengst et al., 2005, J. Bact. 187(2):512-521; and Golic et al., 2005, Microbiology 151:439-446. Other lactic acid bacterial promoter useful for the expression vectors will be apparent to the skilled artisan.

In some embodiments, the promoter used in the recombinant lactic acid bacterium is a regulatable or inducible promoter. The factor(s) regulating or inducing the promoter include any physical and chemical factor that can regulate the activity of a promoter sequence, including, but not limited to, physical conditions, such as temperature and light; chemical substances, such as IPTG, tryptophan, lactate or nisin; and environmental or growth condition factors, such as pH, incubation temperature, and oxygen content. Other conditions for regulating promoter activity can include, among others, a temperature shift eliciting the expression of heat shock genes; the composition of the growth medium such as the ionic strength/NaCl content; accumulation of metabolites, including lactic acid/lactate, intracellularly or in the medium; the presence/absence of essential cell constituents or precursors therefore; and the growth phase or growth rate of the bacterium (see, e.g., U.S. Publication 2002/0137140, the disclosure of which is incorporated herein by reference in its entirety).

A number of inducible gene expression systems for use in lactic acid bacteria have been developed (see, e.g., Kok, 1996, Antonie Van Leeuwenhoek. 70:129-145; Kuipers et al., 1997, Trends Biotechnol. 15:135-40; Djordjevic and Klaenhammer, 1998, Mol. Biotechnol. 9:127-139; Kleerebezem, et al., 1997, Appl Environ Microbiol. 63:4581-4584). An example of a lactic acid bacterial inducible expression system is a system based on the lac promoter transcribing the lac genes of Lactococcus lactis. The lac promoter can be repressed by the LacR repressor, and a six-fold induction of transcription can be obtained by replacing glucose in the growth medium with lactose (van Rooijen et al., 1992, J. Bacteriol. 174(7):2273-80). This naturally occurring expression system has been combined with the T7 RNA polymerase/T7 promoter system from E. coli (Steidler et al., 1995, Appl Environ Microbiol. 61(4): 1627-9). The lac promoter controls the expression of T7 RNA polymerase, which recognizes the T7 promoter, allowing inducible expression of genes cloned downstream of the T7 promoter. In some embodiments, the inducible promoter is the dnaJ promoter transcribing the dnaJ gene of L. lactis, which has been used to generate inducible expression of a heterologous protein after heat shock induction (van Asseldonk et al., 1993, J. Bacteriol. 175(6):1637-44). Increasing the temperature from 30° C. to 42° C. can result in about four-fold induction of gene transcription using the dnaJ system.

Another useful lactic acid bacterial inducible expression systems is the NICE system (de Ruyter et al., 1996, Appl Environ Microbiol. 62:3662-3667), which is based on genetic elements from a two-component system that controls the biosynthesis of the anti-microbial peptide nisin in L. lactis. In some embodiments, inducible expression systems can use genetic elements from the following systems: (1) L. lactis bacteriophages φ31 (O'Sullivan et al., 1996, Biotechnology (NY) 14:82-87; and Walker and Klaenhammer, 1998, J. Bacteriol. 180:921-931) and bacteriophage rlt (Nauta et al., 1997, Nat. Biotechnol. 15:980-983); (2) promoters regulatable by changes in the environment such as pH (Israelsen et al., 1995, Appl Environ Microbiol. 61:2540-2547); (3) metal regulatable promoters, such as Zn²⁺ inducible promoters (Llull and Poquet, 2004, Appl Environ Microbiol. 70:5398-5406); (4) promoters regulatable by salt concentration (Sanders et al., 1998, Mol Gen Genet, 257:681-685); and (5) promoters regulatable by metabolites produced by the host cell.

In some embodiments, the regulatable promoter comprises a pH (e.g., acid) inducible promoter. An exemplary promoter of this type is the pH and growth phase-dependent promoter P170 of L. lactis, as described in WO 94/16086, WO 98/10079, U.S. application publication No. 2002/0137140, and Madsen et al., 1999, Mol. Microbiol. 107:75-87; incorporated herein by reference. The minimal P170 promoter region contains an extended −10 promoter sequence but not a consensus −35 sequence. This non-canonical −35 region has also been observed in other L. lactis promoters (Walker and Klaenhammer, 1998, J. Bacteriol. 180(4):921-31). In the P170 promoter, a 27 bp DNA segment located 15 bp upstream of the extended −10 region of the promoter is responsible for the pH and growth phase regulated promoter activity.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

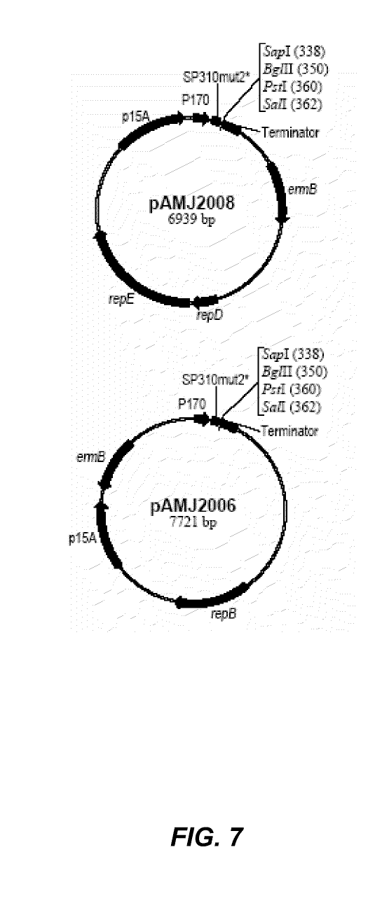

In some embodiments, the promoter and the polynucleotide sequence coding for the apolipoprotein can be introduced into a Gram-positive bacterium on an autonomously replicating replicon, the replication of which is independent of chromosomal replication, e.g., a plasmid, transposable element, bacteriophage, a minichromosome, or an artificial chromosome (see, e.g., U.S. Pat. No. 5,580,787). For autonomous replication, the vector may comprise an origin of replication enabling the vector to replicate autonomously in the host cell. Examples of bacterial origins of replication include, among others, P15A ori (as shown in the plasmid of FIG. 7) or the origins of replication of plasmids pBR322, pUC19, pACYC177 (which plasmid has the P15A ori), and pACYC184, permitting replication in E. coli; and pUB110, pE194, pTA1060, or pAMβ1 permitting replication in Bacillus.

In some embodiments, the recombinant nucleic acid can be introduced under conditions in which the apolipoprotein polynucleotide coding sequence becomes integrated into the Gram-positive bacterium cell chromosome, so as to provide stable maintenance in the bacterium of the apolipoprotein nucleotide coding sequence. Integration can be effected by integration systems based on, among others, homologous recombination, transposons, conjugal transfers, and phage integrases (see, e.g., Frazier et al., 2003, Appl Environ Microbiol. 69(2):1121-8; Christiansen et al., 1994, J. Bacteriol. 176(4):1069-76; Romero et al., 1992, Appl Environ Microbiol. 58(2):699-702; Romero et al., 1991, J. Bacteriol. 173(23):7599-606; Leenhouts et al., 1991, Appl Environ Microbiol. 57(9):2562-7; Leenhouts et al., 1990, Appl Environ Microbiol. 56(9):2726-2735; Chopin et al., 1989, Appl Environ Microbiol. 55(7):1769-74; and Scheirlinck et al., 1989, Appl Environ Microbiol. 55(9):2130-7). In some embodiments, the apolipoprotein nucleotide coding sequence can be introduced into the Gram-positive bacterium cell chromosome at a location where it becomes operably linked to a promoter naturally occurring in the chromosome of the selected host organism (see, e.g., Rauch et al., 1992, J. Bacteriol. 174(4):1280-7; Israelsen et al., 1993, Appl Environ Microbiol. 59(1):21-26; and Maguin et al., 1996, J. Bacteriol. 178(3):931-5).

In addition to the above sequences which form part of the expression vector, the vector may also comprise a selectable marker allowing the stable maintenance of the vector in a host cell and selection of transformants. The choice of a suitable marker will depend on the particular use of the vector, and the choice can readily be made by those skilled in the art. Examples of useful selectable markers include complementable auxotrophy markers or genes mediating resistance to heavy metals, antibiotics or bacteriocins. Exemplary markers that confer antibiotic resistance include, among others, ampicillin, kanamycin, chloramphenicol, tetracycline resistance genes, or the DELPHI® system.

In some embodiments, additional nucleotide sequences, such as those used to improve the production and secretion of heterologous proteins in lactic acid bacteria can be used in the methods and compositions described herein. For example, in some embodiments, nucleotide sequences coding for staphylococcal nuclease (Nuc) and the synthetic propeptide LEISSTCDA, can be linked to a nucleotide sequence coding for an apolipoprotein (see, e.g., Nouaille et al., 2005, Braz J Med Biol Res. 38:353-359, the disclosure of which is incorporated herein by reference).

In some embodiments, the expression vectors are those used in lactic acid bacteria to express heterologous polypeptides in a lactic acid bacterium. Exemplary lactic acid bacteria expression vectors include, but are not limited to, pLF22 (see, e.g., Trakanov, et al., 2004, Microbiology 73:170-175) and pTREX (see, e.g., Reuter, et al., 2003, “Vaccine Protocols,” in Methods in Molecular Medicine 87:101-114).

The recombinant nucleic acids and corresponding expression vectors for expressing secreted apolipoprotein can be prepared by methods well known in the art. Guidance is provided in Sambrook et al., 2001, Molecular Cloning: A Laboratory Manual, 3^{rd} Ed., Cold Spring Harbor Laboratory Press; and Current Protocols in Molecular Biology, Ausubel. F. ed., Greene Pub. Associates, 1998, updates to 2007. Where applicable, polynucleotides encoding the signal peptide and apolipoprotein can be can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the present disclosure can be prepared by chemical synthesis using, e.g., the classical phosphoramidite method described by Beaucage et al., 1981, Tet Lett 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, Tex., The Great American Gene Company, Ramona, Calif., ExpressGen Inc. Chicago, Ill., Operon Technologies Inc., Alameda, Calif., and many others. In some embodiments, oligonucleotide primers can be used to synthesize the desired polynucleotide using polymerase chain reaction in presence of an appropriate polynucleotide template.

The expression vectors can be introduced into the host cells by a variety techniques such that the nucleic acid can replicate, either as an extrachromosomal element or chromosomal integrant. Exemplary methods for transformation include, among others, CaCl₂ (Mandel and Higa, 1970, J Mol Biol 53:159-162), electroporation (Miller et al., 1988, Proc Natl Acad Sci USA 85:856-860; Shigekawa and Dower, 1988, BioTechnique 6:742-751; Ausubel et al., 1995, Current Protocols in Molecular Biology, Unit 9.3, John Wiley & Sons, Inc.); DEAE-dextran (Lopata et al., 1984, Nucleic Acids Res. 12:5707), liposome-mediated transfection (Felgner et al., 1987, Proc Natl Acad. Sci. USA 84:7413-7417); biolistic particle bombardment (see, e.g., Sanford et al., 1987, J. Particle Sci. Technol. 5:27-37); and protoplast transformation (see, e.g., Chang and Cohen, 1979, Mol. Gen. Genet. 168:111-115). Other methods for introducing the expression vectors will be apparent to the skilled artisan.

### 5.6 HOST CELLS AND LACTIC ACID BACTERIA

In various embodiments, the recombinant nucleic acid and the expression vectors comprising the nucleic acid is introduced into a host cell to generate recombinant host cells expressing an apolipoprotein. Various host cells can be used, including Gram-positive and Gram-negative bacteria. For producing endotoxin free apolipoproteins, host cells which are gram-positive bacteria, such as lactic acid bacteria, can be used. As used herein, the term “lactic acid bacterium” refers to a gram-positive, microaerophilic or anaerobic bacterium that ferments sugars with the production of acids, including lactic acid as the predominantly produced acid. Typically, the methods and compositions employ lactic acid bacteria that are used industrially, such as Lactococcus spp., Streptococcus spp., Lactobacillus spp., Leuconostoc spp., Pediococcus spp., Brevibacterium spp. and Propionibacterium spp. Lactic acid producing bacteria belonging to the strictly anaerobic group, bifidobacteria, i.e., Bifidobacterium spp., which are frequently used as food starter cultures alone or in combination with lactic acid bacteria, can also be included within the lactic acid bacteria family.

In some embodiments, the host cells used to generate the recombinant lactic acid bacterium may be selected from Lactococcus spp. including Lactococcus lactis spp. lactis, Lactococcus lactis spp. diacetylactis and Lactococcus lactis spp. cremoris, Streptococcus spp. including Streptococcus salivarius spp. thermophilus, Lactobacillus spp. including Lactobacillus acidophilus, Lactobacillus plantarum, Lactobacillus delbruckii spp. bulgaricus, Lactobacillus helveticus, Leuconostoc spp. including Leuconostoc oenos, Pediococcus spp., Brevibacterium spp., Propionibacterium spp. and Bifidobacterium spp. including Bifidobacterium bifidum.

Lactococcus lactis is commonly used in the production of fermented dairy products such as cheese, sour cream and buttermilk, and has been adapted for producing recombinant proteins and as a vaccine delivery vehicle (Wells et al., 1996, Antonie Van Leeuwenhoek. 70(2-4):317-30; Kuipers et al., 1997, Trends Biotechnol. 15(4):135-40). In addition to the various references describe above, molecular techniques for manipulation of lactic acid bacterium are described in, among others, Dieye et al., 2001, J. Bacteriol. 183(14): 4157-4166; Genetics of Lactic Acid Bacteria, (Wood and Warner Eds.) Springer (2003); Kok, J. 1996, Antonie Van Leeuwenhoek. 70(2-4):129-45; and de Vos W M., 1999, Curr Opin Microbiol. 2(3):289-95; the disclosures of which are incorporated herein by reference.

It is to be understood that other non-endotoxin producing bacteria, including other gram-positive bacteria known to those of skill in the art, can be used to produce recombinant apolipoproteins such that the scope of production of recombinant apolipoproteins is not limited to the lactic acid bacteria described above. In some embodiments, the gram-positive microorganism can be a member of Streptomyces or Bacillus. Host cells of the Bacillus family that can be useful for expressing the apolipoprotein include, among others, B. licheniformis, B. lentus, B. brevis, B. stearothermophilus, B. alkalophilus, B. amyloliquefaciens, B. coagulans, B. circulans, B. lautus, B. thuringiensis, B. methanolicus and B. anthracis.

In some embodiments, the gram-positive bacterium used to express a recombinant apolipoprotein can be a variant host cell deficient in one more intracellular or extracellular proteases (see, e.g., Pritchard and Coolbear, 1993, FEMS Microbiol Rev 12: 179-206; Stefanitsi et al., 1997, Lett Appl Microbiol 24:180-184; and Smeds et al., 1998, J Bacteriol 180:6148-6153). Deficiency or absence of the proteases can limit adverse proteolytic processing of the expressed polypeptides, thereby enhancing the level of intact polypeptides produced by the host cell. In some embodiments, the host cell is deficient in the extracellular housekeeping protease represented by HtrA. Lactic acid bacterial strain with an inactivated HtrA gene is described in Miyoshi et al., 2002, Appl Environ Microbiol. 68:3141-3146 and Poquet et al., 2000, Mol. Microbiol. 35(5):1042-1051, the disclosures of which are incorporated herein by reference in its entirety. In some embodiments, the host cell is deficient in the extracellular serine protease represented by PrtP, which is described in Pritchard and Coolbear, supra, and Kunji et al., 1996, Antonie van Leeuwenhoek 70:187-221. In some embodiments, the host cell is deficient in the protease N is P, a transposon-encoded protease that processes the nisin precursor after its secretion, to release the fully mature nisin and bacteriocin (see, e.g., van der Meer et al., 1993, J Bacteriol 175:2578-2588). In some embodiments, the host cell is deficient in a combination of proteases, such as the combination of represented by HtrA and PrtP proteases (Poquet et al., supra). Host cells deficient in other proteases affecting production of heterologous proteins will be apparent to the skilled artisan.

### 5.7 GROWTH OF HOST CELLS AND PREPARATION OF APOLIPOPROTEIN

The present disclosure further provides methods of producing apolipoprotein by culturing the host cells comprising the recombinant polynucleotides and/or expression vectors under conditions where the fusion polypeptide is expressed and secreted from the host cell. One of ordinary skill in the art is competent to select appropriate culturing conditions. The production of the fusion polypeptide and apolipoprotein can be monitored in any of a number of ways that will be apparent to those skilled in the art and as described above. In some embodiments, the Gram-positive host cell comprising the recombinant nucleic acid herein can be cultivated, for example as disclosed in U.S. Publication 2002/0137140, to produce endotoxin-free apolipoprotein.

The terms “cultivation” or “culturing” are used interchangeably to refer to a cultivation technique where one or more nutrients are supplied during cultivation to the culturing container or bioreactor and in which the cultivated cells and the gene product remain in the container or bioreactor. In some embodiments, nutrients can be fed to the culturing container to provide “continuous cultivation.” In continuous culturing or cultivation, nutrients are continuously added to the cultivation container or bioreactor and fractions of the medium and/or cell culture removed at the same flow rate as that of supplied nutrients to maintain a constant culture volume. In some embodiments, the host cells are cultured in a conventional “batch” process where all nutrients needed during a culturing run are present in the culturing container or bioreactor before cultivation is started, except for, in some embodiments, molecular oxygen in an aerobic process and chemicals for pH adjustment.

In some embodiments, the recombinant host cell is cultivated in a defined medium. “Defined medium” refers to nutrient medium that essentially does not contain undefined nitrogen or carbon sources (e.g., animal or plant protein or protein hydrolysate compositions or complex carbon sources) but rather where the nitrogen sources are well-defined inorganic or organic compounds such as ammonia or amino acids, and the carbon source is a well-defined sugar such as glucose. Additionally, the synthetic medium can contain mineral components such as salts, e.g. sulfates, acetates, phosphates and chlorides of alkaline and earth alkaline metals, vitamins and micronutrients. In some embodiments, the media used to culture the host cells is undefined medium, the compositions of which are well known to the skilled artisan.

The culture medium generally contains a carbon source, the type and concentration of which depends on the type of recombinant host bacterium used (e.g., lactic acid bacterium) and the selected cultivation conditions. Exemplary carbon sources include, but are not limited to, glucose, lactose, and galactose. The appropriate amount of carbon source in the media can be readily determined by those skilled in the art. For example, glucose can be at concentration of at least about 0.5 g/L by controlled feeding of glucose or by feeding of glucose-containing complete medium in a continuous cultivation process. In some embodiments, the glucose concentration in the cultivation medium may be at least 5 g/L, or at least 10, 15, 20, 30, 40, 50, 80 or 100 g/L. A person of skill in the art will be able to formulate other media conditions that permit culturing of recombinant host cells for producing apolipoprotein.

Generally, the culturing conditions, such as time, temperature, pH, and aeration conditions, if relevant, and the rate of nutrient addition can depend on the particular type of host cell bacterium used. An exemplary culturing condition is for 24-72 hours at a temperature in the range of 15-40° C. and at a pH in the range of 4-8. Continuous culturing process may run for longer periods of time as desirable, such as several hundred hours.

In some embodiments, the culturing conditions can further comprise removal of media components during the culturing process to enhance production of the desired polypeptide. In some embodiments where the host cell is a lactic acid bacterium, the culturing conditions comprise continuous removal of the lactic acid formed during the culturing process. This can be done by chromatographic techniques, or reverse electro-enhanced dialysis (REED® systems; Jurag Separation, Alleroed, Denmark), as described in WO 02/48044, incorporated herein by reference. The REED® system allows control of pH in the fermentation broth by exchanging low-molecular weight negatively charged molecules in the fermentation broth with hydroxide ions. Other methods for removal of lactic acid include, among others, recirculation of cells back to the fermentor following separation from media during culturing, and use of ammonium or calcium phosphate to titrate the pH.

In some embodiments, short chain acyl phospholipids can be added to the medium used to culture the recombinant lactic acid bacteria. The lactic acid bacteria can utilize the short chain acyl phospholipids as a nutrient source. Additionally, the short chain acyl phospholipids can aid in solubilizing the expressed apolipoprotein. The short chain acyl phospholipids are easily removed by adding a phospholipase that hydrolyzes the acyl chain, liberating a short chain fatty acid and a short chain lysoPL. As the short chain fatty acid and lysoPL are soluble, the apolipoprotein can be precipitated and purified.

In some embodiments, the recombinant host cell can be cultivated to express the desired apolipoprotein, and the polypeptide harvested using conventional techniques for separating cells, polypeptides, either during the culturing (e.g., when continuous) or when the culturing step is terminated. The expressed apolipoproteins can be recovered from the cells and/or the culture medium using any one or more of the well known techniques for protein isolation and purification, including, among others, lysozyme treatment, sonication, filtration, salting-out, ultra-centrifugation, and chromatography.

Chromatographic techniques for isolation of the expressed polypeptide include, among others, reverse phase chromatography high performance liquid chromatography, ion exchange chromatography, gel electrophoresis, and affinity chromatography. Conditions for purifying the apolipoprotein will depend, in part, on factors such as net charge, hydrophobicity, hydrophilicity, molecular weight, molecular shape, etc., and will be apparent to those having skill in the art. Exemplary method for harvesting recombinant cells and recovering the apolipoprotein are described in, for example, U.S. application publication No. 2002/0137140, the disclosure of which is incorporated herein by reference in its entirety.

In some embodiments, affinity techniques can be used to isolate the expressed polypeptides. For affinity chromatography purification, any antibody which specifically binds the polypeptide (e.g., signal peptide and/or apolipoprotein) may be used. For the production of antibodies, various host animals, including but not limited to rabbits, mice, rats, etc., may be immunized by injection with the polypeptide. The immunogen may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, and potentially useful human adjuvants such as BCG (bacilli Calmette Guerin) and Corynebacterium parvum.

### 5.8 PREPARATION OF APOLIPOPROTEIN-LIPID COMPLEXES

The recombinant apolipoproteins described herein can be used for any purpose the polypeptides have been shown to be useful, such as for therapeutic applications in treating or preventing dyslipidemia and/or any disease, condition and/or disorder associated with dyslipidemia. In such applications, the apolipoproteins can be formulated and administered in an apolipoprotein-lipid complex.

In various embodiments, the recombinant apolipoproteins can be complexed with a variety of lipids, including saturated, unsaturated, natural and synthetic lipids and/or phospholipids. Suitable lipids include, but are not limited to, small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, sphingomyelin, sphingolipids, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, dipalmitoylsphingomyelin, distearoylsphingomyelin, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives.

A variety of methods well known to those skilled in the art can be used to prepare the apolipoprotein-lipid vesicles or complexes. To this end, a number of available techniques for preparing liposomes or proteoliposomes can be used. For example, the apolipoprotein can be cosonicated (e.g., using a sonic bath or probe sonicator) with appropriate lipids to form complexes. Alternatively the apolipoprotein can be combined with preformed lipid vesicles resulting in the spontaneous formation of apolipoprotein-lipid complexes. In some embodiments, the apolipoprotein-lipid complexes can be formed by a detergent dialysis method, a process in which a mixture of the apolipoprotein, lipid and detergent is dialyzed to remove the detergent and reconstituted to form apolipoprotein-lipid complexes (see, e.g., Jonas et al., 1986, Methods Enzymol. 128:553-582).

Another method for preparing apolipoprotein-phospholipid complexes which have characteristics similar to HDL is described in U.S. Pat. No. 6,004,925, the disclosure of which is incorporated herein by reference. The lyophilized product can be reconstituted in order to obtain a solution or suspension of the peptide-lipid complex. For reconstitution, the lyophilized powder is rehydrated with an aqueous solution to a suitable volume (e.g., 5 mg peptide/ml, which is convenient for intravenous injection). In some embodiments, the lyophilized powder is rehydrated with phosphate buffered saline or a physiological saline solution. The mixture can be agitated or vortexed to facilitate rehydration. The reconstitution step can be conducted at a temperature equal to or greater than the phase transition temperature of the lipid component of the complexes.

In other embodiments, recombinant apolipoprotein-lipid complexes are made by complexing the recombinant apolipoproteins with the lipids disclosed in U.S. application publication No. 20060217312 and International application publication No. WO/2006/100567 (PCT/IB2006/000635), the disclosures of which are incorporated herein by reference.

In some embodiments, co-lyophilization methods commonly known in the art are used to prepare the polypeptide-lipid complexes. Briefly, the co-lyophilization steps include solubilizing the apolipoprotein and lipid in organic solvent of solvent mixture, or solubilizing apolipoprotein and lipid separately and mixing them together. The desirable characteristics of solvent or solvent mixture are: (i) a medium relative polarity to be able to dissolve hydrophobic lipids and amphipatic protein, (ii) solvents should be class 2 or 3 solvent according to FDA solvent guidelines (Federal Register, volume 62, No. 247) to avoid potential toxicity associated with the residual organic solvent, (iii) low boiling point to assure ease of solvent removal during lyophilization, (iv) high melting point to provide for faster freezing, higher temperatures of condenser and, hence less ware of freeze-dryer. In some embodiments, glacial acetic acid is used. Combinations of methanol, glacial acetic acid, xylene, or cyclohexane may also be used.

The apolipoprotein-lipid solution is then lyophilized to obtain a homogeneous powder. The lyophilization conditions can be optimized to obtain fast evaporation of solvent with minimal amount of residual solvent in the lyophilized apolipoprotein-lipid powder. The selection of freeze-drying conditions can be determined by the skilled artisan, depending on the nature of solvent, type and dimensions of the receptacle, holding solution, fill volume, and characteristics of freeze-dryer used.

The apolipoprotein-lipid complexes can form spontaneously after hydration of apolipoprotein-lipid lyophilized powder with an aqueous media of appropriate pH and osmolality. In some embodiments, the media may also contain stabilizers such as sucrose, trehalose, glycerin and others. In some embodiments, the solution must be heated several times above transition temperature for lipids for complexes to form. The ratio of lipid to protein can be from 1:1 to 200:1 (mole/mole), and is preferably 2:1 weight of lipid to weight of protein (wt/wt). Powder is hydrated to obtain final complex concentration of 5-30 mg/ml expressed in protein equivalents.

In some embodiments, apolipoprotein powder can be obtained by freeze-drying the polypeptide solution in NH₄HCO₃ aqueous solution. A homogeneous solution of apolipoprotien and lipid (e.g., sphingomyelin) is formed by dissolving their powders and the polypeptide in glacial acetic acid. The solution is then lyophilized, and HDL-like apolipoprotein-lipid complexes formed by hydration of lyophilized powder with aqueous media.

In some embodiments, apolipoprotein-lipid complexes can be formed by co-lyophilization of phospholipid with peptide or protein solutions or suspensions. The homogeneous solution of polypeptide and lipid (e.g., phospholipids) in an organic solvent or organic solvent mixture can be lyophilized, and apolipoprotein-lipid complexes formed spontaneously by hydration of the lyophilized powder with an aqueous buffer. Examples of organic solvents or their mixtures are include, but are not limited to, acetic acid, acetic acid and xylene, acetic acid and cyclohexane, and methanol and xylene.

An aliquot of the resulting reconstituted preparations can be characterized to confirm that the complexes have the desired size distribution; e.g., the size distribution of HDL. An exemplary method for characterizing the size is gel filtration chromatography. A series of proteins of known molecular weight and Stokes' diameter, as well as human HDL, can be used as standards to calibrate the column.

Protein and lipid concentration of apolipoprotein-lipid particles in solution can be measured by any method known in the art, including, but not limited to, protein and phospholipid assays as well as by chromatographic methods such as HPLC, gel filtration chromatography, GC coupled with various detectors including mass spectrometry, UV or diode-array, fluorescent, elastic light scattering and others. The integrity of lipid and proteins can be also determined by the same chromatographic techniques as well as by peptide mapping, SDS-page gel electrophoresis, N- and C-terminal sequencing of proteins, and standard assays for determining lipid oxidation.

### 5.9 THERAPEUTIC AND OTHER USES OF THE APOLIPOPROTEIN

The apolipoprotein-lipid complexes made from the apolipoproteins described herein can be used to treat or prevent a disease, condition or disorder responsive to apolipoproteins or other apolipoprotein-phospholipid particles (e.g., ApoAI-Soybean PC, ApoAI-POPC). In some embodiments, the complexes and compositions can be used to treat or prevent dyslipidemia and/or any disease, condition and/or disorder associated with dyslipidemia. As used herein, the terms “dyslipidemia” or “dyslipidemic” refer to an abnormally elevated or decreased level of lipid in the blood plasma, including, but not limited to, the altered level of lipid associated with the following conditions: coronary heart disease; coronary artery disease; cardiovascular disease; hypertension; restenosis; vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein.

Diseases associated with dyslipidemia include, but are not limited to coronary heart disease, coronary artery disease, acute coronary syndrome, cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein.

In some embodiments, the methods herein encompass treating or preventing a disease associated with dyslipidemia, comprising administering to a subject a recombinant apolipoprotein and/or recombinant apolipoprotein-lipid complex in an amount effective to achieve a serum level of free or complexed apolipoprotein for at least one day following administration that is in the range of about 10 mg/dL to 300 mg/dL higher than a baseline (initial) level prior to administration.

In some embodiments, the methods encompass a method of treating or preventing a disease associated with dyslipidemia, comprising administering to a subject a recombinant apolipoprotein and/or recombinant apolipoprotein-lipid complex in an amount effective to achieve a circulating plasma concentrations of a HDL-cholesterol fraction for at least one day following administration that is at least about 10% higher than an initial HDL-cholesterol fraction prior to administration.

In some embodiments, the methods encompass a method of treating or preventing a disease associated with dyslipidemia, comprising administering to a subject a charged lipoprotein complex or composition described herein in an amount effective to achieve a circulating plasma concentration of a HDL-cholesterol fraction that is between 30 and 300 mg/dL between 5 minutes and 1 day after administration.

In some embodiments, the methods encompass a method of treating or preventing a disease associated with dyslipidemia, comprising administering to a subject a recombinant apolipoprotein and/or recombinant apolipoprotein-lipid complex in an amount effective to achieve a circulating plasma concentration of cholesteryl esters that is between 30 and 300 mg/dL between 5 minutes and 1 day after administration.

In some embodiments, the methods encompass a method at treating or protecting a disease associated with dyslipidemia, comprising administering to a subject a recombinant apolipoprotein and/or recombinant apolipoprotein-lipid complex in an amount effective to achieve an increase in fecal cholesterol excretion for at least one day following administration that is at least about 10% above a baseline (initial) level prior to administration.

The recombinant apolipoprotein and/or recombinant apolipoprotein-lipid complexes or compositions described herein can be used alone or in combination therapy with other drugs used to treat or prevent the foregoing conditions. Such therapies include, but are not limited to simultaneous or sequential administration of the drugs involved. For example, in the treatment of hypercholesterolemia or atherosclerosis, recombinant apolipoproteins and/or recombinant apolipoprotein-lipid complexes can be administered with any one or more of the cholesterol lowering therapies currently in use; e.g., bile-acid resins, niacin, statins, inhibitors of cholesterol absorption and/or fibrates. Such a combined regimen can produce particularly beneficial therapeutic effects since each drug acts on a different target in cholesterol synthesis and transport; i.e., bile-acid resins affect cholesterol recycling, the chylomicron and LDL population; niacin primarily affects the VLDL and LDL population; the statins inhibit cholesterol synthesis, decreasing the LDL population (and perhaps increasing LDL receptor expression); whereas the charged lipoprotein complexes described herein affect RCT, increase HDL, and promote cholesterol efflux.

In some embodiments, the recombinant apolipoproteins and/or recombinant apolipoprotein-lipid complexes can be used in conjunction with fibrates to treat or prevent coronary heart disease; coronary artery disease; cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein.

For the various therapeutic uses described herein, the apolipoprotein and/or apoliprotein-lipid complexes can be formulated in a pharmaceutical composition comprising the recombinant apoliprotein as described herein, or the recombinant apolipoprotein-lipid complex as the active ingredient with a pharmaceutically acceptable carrier suitable for administration and delivery in vivo. In embodiments using peptide mimetic apolipoproteins, the peptide mimetic apolipoproteins can be included in the compositions in either the form of free acids or bases, or in the form of pharmaceutically acceptable salts. Modified proteins such as amidated, acylated, acetylated or pegylated proteins, can also be used.

Injectable compositions include sterile suspensions, solutions or emulsions of the active ingredient in aqueous or oily vehicles. The compositions can also comprise formulating agents, such as suspending, stabilizing and/or dispersing agent. The compositions for injection can be presented in unit dosage form, e.g., in ampules or in multidose containers, and can comprise added preservatives. For infusion, a composition can be supplied in an infusion bag made of material compatible with charged lipoprotein complexes, such as ethylene vinyl acetate or any other compatible material known in the art.

Alternatively, the injectable compositions can be provided in powder form for reconstitution with a suitable vehicle, including but not limited to, sterile pyrogen free water, buffer, dextrose solution, etc., before use. For these purposes, the recombinant apolipoprotein can be lyophilized, or prepared as co-lyophilized apolipoprotein-lipid complexes. The stored compositions can be supplied in unit dosage forms and reconstituted prior to use in vivo.

For prolonged delivery, the active ingredient can be formulated as a depot composition for administration by implantation, such as by subcutaneous, intradermal, or intramuscular injection. Thus, for example, recombinant apolipoprotein-lipid complex or recombinant apolipoprotein alone can be formulated with suitable polymeric or hydrophobic materials (e.g., as an emulsion in an acceptable oil) or in phospholipid foam or ion exchange resins.

Alternatively, transdermal delivery systems manufactured as an adhesive disc or patch that slowly releases the active ingredient for percutaneous absorption can be used. To this end, permeation enhancers can be used to facilitate transdermal penetration of the active ingredient. A particular benefit can be achieved by incorporating the charged complexes described herein into a nitroglycerin patch for use in patients with ischemic heart disease and hypercholesterolemia. In some embodiments, the delivery can be done locally or intramurally (within the vessel wall) using a catheter or perfusor (see, e.g., U.S. application publication No. 20030109442).

The compositions can, if desired, be presented in a pack or dispenser device that may comprise one or more unit dosage forms comprising the active ingredient. The pack can, for example, comprise metal or plastic foil, such as a blister pack. The pack or dispenser device can be accompanied by instructions for administration.

The recombinant apolipoproteins and/or recombinant apolipoprotein-lipid complexes can be administered by any suitable route that ensures bioavailability in the circulation. For example, the recombinant apolipoproteins and/or recombinant apolipoprotein-lipid complexes can be administered in dosages that increase the small HDL fraction, for example, the pre-beta, pre-gamma and pre-beta-like HDL fraction, the alpha HDL fraction, the HDL3 and/or the HDL2 fraction. In some embodiments, the dosages are effective to achieve atherosclerotic plaque reduction as measured by, for example, imaging techniques such as magnetic resonance imaging (MRI) or intravascular ultrasound (IVUS). Parameters to follow by IVUS include, but are not limited to, change in percent atheroma volume from baseline and change in total atheroma volume. Parameters to follow by MRI include, but are not limited to, those for IVUS and lipid composition and calcification of the plaque. The plaque regression can be measured using the patient as its own control, time zero versus time t at the end of the last infusion, or within weeks after the last infusion, or within 3 months, 6 months, or 1 year after the start of therapy.

Administration can be achieved by parenteral routes of administration, including intravenous (IV), intramuscular (IM), intradermal, subcutaneous (SC), and intraperitoneal (IP) injections. In certain embodiments, administration is by a perfusor, an infiltrator or a catheter. In some embodiments, the charged lipoprotein complexes are administered by injection, by a subcutaneously implantable pump, or by a depot preparation, in amounts that achieve a circulating serum concentration equal to that obtained through parenteral administration. The complexes could also be absorbed in, for example, a stent or other device.

Administration can be achieved through a variety of different treatment regimens. For example, several intravenous injections can be administered periodically during a single day, with the cumulative total volume of the injections not reaching the daily toxic dose. Alternatively, one intravenous injection can be administered about every 3 to 15 days, preferably about every 5 to 10 days, and most preferably about every 10 days. In yet another alternative, an escalating dose can be administered, starting with about 1 to 5 doses at a dose between (50-200 mg) per administration, then followed by repeated doses of between 200 mg and 1 g per administration. Depending on the needs of the patient, administration can be by slow infusion with a duration of more than one hour, by rapid infusion of one hour or less, or by a single bolus injection.

In some embodiments, administration can be done as a series of injections and then stopped for 6 months to 1 year, and then another series started. Maintenance series of injections can then be administered every year or every 3 to 5 years. The series of injections could be done over a day (perfusion to maintain a specified plasma level of complexes), several days (e.g., four injections over a period of eight days) or several weeks (e.g., four injections over a period of four weeks), and then restarted after six months to a year.

Other routes of administration can be used. For example, absorption through the gastrointestinal tract can be accomplished by oral routes of administration, including but not limited to ingestion, buccal and sublingual routes, provided that appropriate formulations are used to avoid or minimize degradation of the active ingredient (e.g., enteric coatings). Alternatively, administration can be via mucosal tissue, for example, vaginal and rectal modes of administration. In some embodiments, the formulations can be administered transcutaneously (e.g., transdermally), or by inhalation.

The actual dose of a recombinant apolipoprotein and/or recombinant apolipoprotein-lipid complex composition administered will depend upon a variety of factors, including, for example, the particular indication being treated, the mode of administration, whether the desired benefit is prophylactic or therapeutic, the severity of the indication being treated and the age and weight of the patient, the bioavailability of the particular active composition, etc. Determining an effective dosage is well within the capabilities of those skilled in the art.

For example, toxicity and therapeutic efficacy of the various recombinant apolipoproteins and/or recombinant apolipoprotein-lipid complexes can be determined using standard pharmaceutical procedures in cell culture or experimental animals for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Recombinant apolipoproteins and/or recombinant apolipoprotein-lipid complexes that exhibit large therapeutic indices are preferred. Non-limiting examples of parameters that can be followed include, among others, liver function indicators, such as the presence of transaminases. The effect on red blood cells could also be monitored, as mobilization of cholesterol from red blood cells causes them to become fragile, or affect their shape.

Patients can be treated from a few days to several weeks before a medical intervention (e.g., preventive treatment), or during or after a medical intervention. Administration can be concomitant to or contemporaneous with another invasive therapy, such as, angioplasty, carotid ablation, rotoblader or organ transplant (e.g., heart, kidney, liver, etc.).

In certain embodiments, recombinant apolipoproteins and/or recombinant apolipoprotein-lipid complexes are administered to a patient whose cholesterol synthesis is controlled by a statin or a cholesterol synthesis inhibitor. In other embodiments, recombinant apolipoproteins and/or recombinant apolipoprotein-lipid complexes are administered to a patient undergoing treatment with a binding resin, e.g., a semi-synthetic resin such as cholestyramine, or with a fiber, e.g., plant fiber, to trap bile salts and cholesterol, to increase bile acid excretion and lower blood cholesterol concentrations.

The recombinant apolipoproteins and/or recombinant apolipoprotein-lipid complexes and compositions described herein can be used in assays in vitro to measure serum HDL, e.g., for diagnostic purposes. Because ApoA-I, ApoA-II and Apo peptides associate with the HDL component of serum, recombinant apolipoproteins and/or recombinant apolipoprotein-lipid complexes can be used as “markers” for the HDL population, and the pre-beta1 and pre-beta2 HDL populations. Moreover, the recombinant apolipoproteins and/or recombinant apolipoprotein-lipid complexes can be used as markers for the subpopulation of HDL that are effective in RCT. For these uses, recombinant apolipoproteins and/or recombinant apolipoprotein-lipid complexes can be added to or mixed with a patient serum sample, and after an appropriate incubation time, the HDL component can be assayed by detecting the incorporated recombinant apolipoproteins and/or recombinant apolipoprotein-lipid complexes. This can be accomplished using labeled recombinant apolipoproteins and/or recombinant apolipoprotein-lipid complexes (e.g., radiolabels, fluorescent labels, enzyme labels, dyes, etc.), or by immunoassays using antibodies (or antibody fragments) specific for recombinant apolipoproteins and/or recombinant apolipoprotein-lipid complexes.

Alternatively, labeled recombinant apolipoproteins and/or recombinant apolipoprotein-lipid complexes can be used in imaging procedures (e.g., CAT scans, MRI scans, etc.) to visualize the circulatory system, monitor RCT, or visualize accumulation of HDL at fatty streaks, atherosclerotic lesions, and the like, where the HDL should be active in cholesterol efflux.

### 6. EXAMPLES

Various features and embodiments of the disclosure are illustrated in the following representative examples, which are intended to be illustrative, and not limiting.

### Example 1

### Codon Optimized Apo-A1 Gene

Codon optimization of the polynucleotide encoding human Apo-A1 was carried out using GeneOptimizer® software of Geneart. The software parameters were set to avoid very high (>80%) or very low (<30%) GC content and avoiding cis acting motifs, including, among others, internal TATA-boxes, chi-sites, and ribosomal entry sites; AT rich or GC rich sequence stretches; repeat sequences; and RNA secondary structure. The sequence of the codon optimized Apo-A1 encoding polynucleotide is shown in FIG. 8.

The codon optimized polynucleotide was synthesized from synthetic oligonucleotides, and the final construct verified by sequencing. The structures of various recombinant polynucleotides with the codon optimized Apo-A1 sequences were constructed and placed into a P170 promoter based expression system, as further described below.

### Example 2

### Cloning and Analysis of Apo-A1 Gene Expression in L. lactis

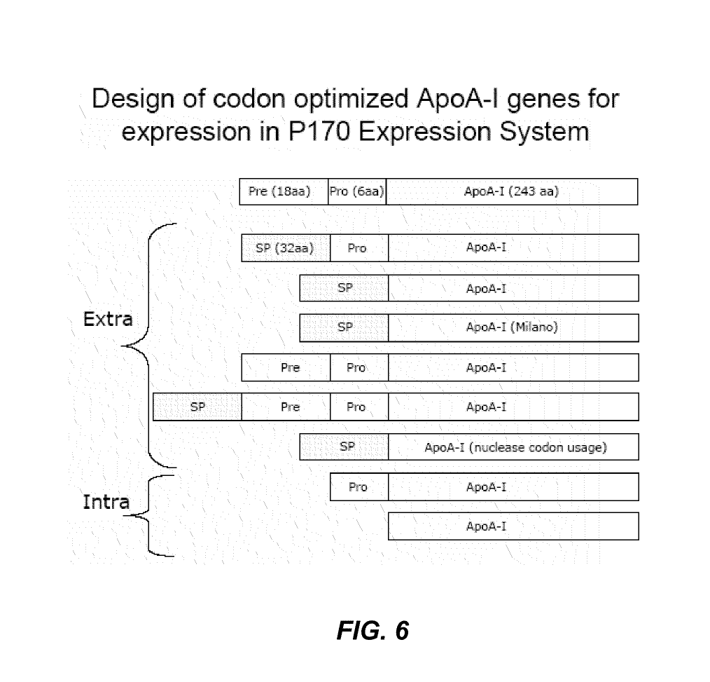

The P170 expression vectors used to test and express Apo-A1 are shown in FIG. 6. Recombinant plasmids established in E. coli are stored at −80° C. in glycerol. The inserted genes were verified by PCR amplification and sequencing of cloned junctions. The constructed expression vectors were used to transform L. lactis strains MG1363 (wt), PSM565, and DOL5. The PSM565 strain is derived from MG1363, and DOL5 is derived from PSM565 using chemical mutagenesis. In general, the strain of interest (MG1363 or PSM565) containing a plasmid encoding an apolipoprotein was mutagenized using ethyl methansulfonate (EMS). EMS was added to a growing culture and samples were withdrawn at selected time points. Those samples were stored at −80° C., later plated on agar plates, and a large number of colonies picked using robotic technology. Supernatants were assayed for increased yield of the reporter gene product encoded by the plasmid. A number of clones that expressed the reporter gene product at a significant higher level compared to the mother strain were selected for further analysis. To confirm that the mutation was carried by the genome and not by the plasmid, selected clones were cured of the plasmid by growing without selective pressure for a number generations. After having cured the clones of interest, the strains were re-transformed with the original plasmid (wild type plasmid) and analyzed for secretion and production of the same reporter gene product. Clones that still showed higher yields were selected (e.g., PSM565 and DOL5) and analyzed for increased yield of ApoA-I.

Three to five colonies from each transformation were isolated, re-streaked and stored at −80° C. in glycerol. The presence of insert was confirmed by PCR in selected isolates. Expression analysis was done in flasks using M17G5 or 2M17G20 medium. Selected isolates were grown for one to two days, and then cell extracts and culture medium supernatant fractions prepared, which were analyzed by SDS-PAGE using Coomassie staining/western blot. ApoA-I was detected using an Apo-A1 specific monoclonal antibody.

### Example 3

### Fermentation and Growth Conditions

Preparation of fermentor inoculum. Bacterial strains are stored in 15-25% (v/v) glycerol at −80° C. The strain of interest is inoculated in 30 ml 2M17G20 (2 times M17 and 2.0% w/v glucose) supplemented with the appropriate antibiotics (e.g., 1 μg/ml of erythromycin) using a plastic inoculation needle and grown for approximately 20 hours at 30° C. (standing culture). The expected OD600 is approximately 3.5-4.0 and pH is approximately 5.0-5.5. Alternatively the strain or interest is inoculated in 30 ml M17G5 broth (0.5% w/v glucose) supplemented with the appropriate antibiotics (e.g., 1 μg/ml of erythromycin) using a plastic inoculation needle and grown for approximately 20 hours at 30° C. (standing culture). The expected OD600 is approximately 3.0 and pH is approximately 5.5.

Fermentation conditions using removal of lactate: Fermentation and growth of the lactic acid bacteria host cells comprising the expression vector used the technology of REED (Reverse Electro-Enhanced Dialysis; see also, WO 02/48044, incorporated herein by reference) (Jurag Separation, Alleroed, Denmark), which allows control of pH in the fermentation broth by exchanging low-molecular weight negatively charged molecules in the fermentation broth with hydroxide ions. The REED process is able to extract small, charged molecules like inorganic and organic acids (e.g., lactate, acetate, etc.) including amino acids, separating these from larger or non-charged components like proteins, sugars, cells, yeast, etc. Thus REED system allows the continuous extraction of organic acids during fermentation.

The protocols for REED-controlled fermentations consisted of three phases. In the first phase, pH is controlled by potassium hydroxide titration. This phase is used for building up cell mass. In the second phase, REED is used to control pH and to keep the lactic acid concentration within a range of 100-150 mM, which allows fast cell growth and keeps the P170 production repressed. When sufficient cell mass/production capacity has built up, the lactate concentration is increased to induce production, and the REED system is used to keep the lactate concentration within the optimal range.

The medium is chemically defined and enriched with yeast extract. Growth conditions are 30° C., pH 6.5 and shaken at 300 rpm for a 1 L fermentor.

In the fed-batch phases, glucose was fed as a 500 g/L solution separately from the concentrated Feed Medium, which contained yeast extract, specific amino acids and some components. The yeast extract is a source of amino acids, oligopeptides, most vitamins and metal ions. Feeding glucose separately allowed the possibility to vary the ratio between the energy source (glucose) and the building blocks for cell growth and product synthesis.

The volumes of medium used were:

| | |
|---|---|
| Start up medium | 0.5 L to 0.8 L |
| Feed medium | 1 L to 1.5 L |
| 500 g/l glucose | 3.4 L |
| 3 M KOH | 59 ml |
| Water | 1.5 L |

Conventional Batch Fermentation. The host cells comprising the expression vectors expressing apolipoprotein were also prepared in a conventional fed-batch fermentation procedure. Conventional fermentation consisting of a fed-batch also resulted in ApoA-I secretion into the medium. The outlines of the fermentation are as follows:

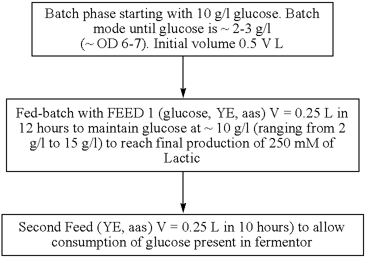

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A recombinant nucleic acid comprising:
(i) a first polynucleotide sequence encoding a signal peptide comprising the structure:
(n)ₓ˜(m)_{y}˜(c)_{z},
wherein
each n is independently any amino acid, with two or more n being a basic amino acid residue.
each m is independently an aromatic, aliphatic, hydrophobic, or hydroxyl containing amino acid residue;
each c is independently any amino acid, with two or more c being a polar amino acid residue;
x is 6, 7, or 8;
y is any integer from 13 to 16; and
z is any integer from 5 to 14;
“˜” is a peptide bond; and
(ii) a second polynucleotide sequence encoding an apolipoprotein,
wherein the first polynucleotide and the second polynucleotide are operably linked to direct secretion of the encoded apolipoprotein.

2. The recombinant nucleic acid of claim 1 in which (n)ₓ comprises the amino acid sequence X¹˜X²˜X³˜X⁴˜X⁵˜X⁶˜X⁷˜X⁸,
wherein
X¹ is M;
X² is a basic amino acid;
X³ is an aromatic amino acid;
X⁴ is a basic or polar amino acid;
X⁵ is a basic amino acid;
X⁶ is a basic amino acid;
X⁷ is a basic amino acid;
X⁸ is an aliphatic amino acid; and
wherein
optionally each of X³ and X⁴ are independently absent.

3. The recombinant nucleic acid of claim 1 in which (m)_{y} comprises the amino acid sequence:
X⁹˜X¹⁰˜X¹¹˜X¹²˜X¹³˜X¹⁴˜X¹⁵˜X¹⁶˜X¹⁷˜X¹⁸˜X¹⁹˜X²⁰˜X²¹˜X²²˜X²³˜X²⁴,
wherein
X⁹ is an aliphatic amino acid; and
X¹⁰ is an aliphatic amino acid;
X¹¹ is an aliphatic amino acid:
X¹² is an aliphatic or hydroxyl containing amino acid;
X¹³ is an aromatic, aliphatic, or hydrophobic amino acid;
X¹⁴ is an aliphatic amino acid;
X¹⁵ is an aliphatic, aromatic, or hydrophobic amino acid;
X¹⁶ is an aliphatic amino acid;
X¹⁷ is an aliphatic amino acid;
X¹⁸ is an aliphatic, aromatic, or hydrophobic amino acid;
X¹⁹ is an aliphatic amino acid;
X²⁰ is an aliphatic, aromatic, hydrophobic, or a hydroxyl containing amino acid;
X²¹ is an aliphatic, aromatic, or hydrophobic amino acid;
X²² is an aliphatic, aromatic, or hydrophobic amino acid;
X²³ is a aliphatic or hydroxyl containing amino acid; and
X²⁴ is an aliphatic amino acid.

4. The recombinant nucleic acid of claim 1 in which (c)_{z} comprises the amino
X²⁵˜X²⁶˜X²⁷˜X²⁸˜X²⁹˜X³⁰˜X³¹˜X³²˜X³³˜X³⁴˜X³⁵˜X³⁶˜X³⁷˜X³⁸,
wherein
X²⁵ is a hydroxyl containing amino acid:
X²⁶ is a hydroxyl containing amino acid;
X²⁷ is an aliphatic amino acid;
X²⁸ is a polar or constrained amino acid residue;
X²⁹ is an acidic amino acid;
X³⁰ is a polar or an aliphatic amino acid;
X³¹ is a polar or hydroxyl containing amino acid;
X³² is an aliphatic or hydroxyl containing amino acid;
X³³ is an polar amino acid;
X³⁴ is an aliphatic amino acid;
X³⁵ is an aliphatic or acidic amino acid;
X³⁶ is an acidic or hydroxyl containing amino acid;
X³⁷ is a basic amino acid; and
X³⁸ is a hydroxyl containing amino acid; and
wherein
optionally each of X²⁵, X²⁶, X²⁸, X²⁹, X³², X³³, X³⁴, X³⁵, X³⁶, X³⁷, and X³⁸ is independently absent.

5. The recombinant nucleic acid of claim 1 in which the encoded signal peptide comprises at least 60% amino acid sequence identity to SEQ ID NO:1.

6. The recombinant nucleic acid of claim 1 in which the encoded signal peptide has at least 80% amino acid sequence identity to SEQ ID NO:1.

7. The recombinant nucleic acid of claim 1 in which the encoded signal peptide has at least 90% amino acid sequence identity to SEQ ID NO:1.

8. The recombinant nucleic acid of claim 1 in which the encoded signal peptide comprises at least 60% amino acid sequence identity to SEQ ID NO:2.

9. The recombinant nucleic acid of claim 1 in which the encoded signal peptide has at least 80% amino acid sequence identity to SEQ ID NO:2.

10. The recombinant nucleic acid of claim 1 in which the encoded signal peptide has at least 90% amino acid sequence identity to SEQ ID NO:2.

11. The recombinant nucleic acid of claim 5 in which the encoded signal sequence comprises one or more amino acid substitutions or deletions at corresponding amino acid residue positions selected from: X³, X⁴, X⁸, X⁹, X¹⁰; X¹¹, X¹², X¹³, X¹⁴, X¹⁵, X¹⁷, X¹⁸, X¹⁹, X²⁰, X²¹, X²², X²³, X²⁵, X²⁶, X²⁸, X²⁹, X³⁰, X³¹, X³², X³³, X³⁵, X³⁶, X³⁷, and X³⁸.

12. The recombinant nucleic acid of claim 11 in which the amino acid substitutions are selected from:
X³ is an aromatic amino acid other than F;
X⁴ is a basic amino acid or polar amino acid other than N;
X⁸ is an aliphatic amino acid other than V;
X⁹ is an aliphatic amino acid other than A;
X¹⁰ is an aliphatic amino acid other than I;
X¹¹ is an aliphatic amino acid other than I
X¹² is an aliphatic amino acid or S;
X¹³ is an aliphatic amino acid or a hydrophobic or aromatic amino acid other than F;
X¹⁴ is an aliphatic amino acid other than I;
X¹⁵ is an aromatic amino acid or a hydrophobic or aliphatic amino acid other than A;
X¹⁷ is an aliphatic amino acid other than I;
X¹⁸ is an aliphatic amino acid or a hydrophobic or aromatic amino acid other than F;
X¹⁹ is an aliphatic amino acid other than V;
X²⁰ is an aliphatic, hydrophobic, aromatic amino acid, or T;
X²¹ is an aliphatic amino acid or a hydrophobic or aromatic amino acid other than F;
X²² is an aliphatic amino acid or a hydrophobic or aromatic amino acid other than F;
X²³ is an aliphatic amino acid or S;
X²⁸ is a constrained amino acid or a polar amino acid other than Q;
X³⁰ is an aliphatic amino acid or polar amino acid other than N;
X³¹ is an hydroxyl containing amino acid or polar amino acid other than Q;
X³² is a hydroxyl containing amino acid or an aliphatic amino acid other than A;
X³³ is a polar amino acid other than N;
X³⁵ is an acidic amino acid or an aliphatic amino acid other than A; and
X³⁶ is a hydroxyl containing amino acid residue or a D.

13. The recombinant nucleic acid of claim 11 in which the encoded signal sequence comprises up to 14 non-conservative substitutions at corresponding amino acid residue positions X⁴, X¹², X¹³, X¹⁵, X¹⁸, X²⁰, X²¹, X²², X²³, X²⁸, X³⁰, X³¹, X³², X³⁵, and X³⁶ and optionally one or more conservative substitutions at other amino acid residue positions.

14. The recombinant nucleic acid of claim 13 in which the amino acid substitutions are selected from:
X⁴ is a basic amino acid;
X¹² is an aliphatic amino acid;
X¹³ is an aliphatic amino acid;
X¹⁵ is an aromatic amino acid;
X¹⁸ is an aliphatic amino acid;
X²⁰ is an aliphatic or aromatic amino acid;
X²¹ is an aliphatic amino acid;
X²² is an aliphatic amino acid;
X²³ is an aliphatic amino acid;
X²⁸ is a constrained amino acid;
X³⁰ is an aliphatic amino acid;
X³¹ is an hydroxyl containing amino acid;
X³² is a hydroxyl containing amino acid;
X³⁵ is an acidic amino acid; and
X³⁶ is a hydroxyl containing amino acid.

15. The recombinant nucleic acid of claim 5 in which amino acid residues are optionally absent at one or more corresponding amino acid residue positions selected from:
X³, X⁴, X²⁵, X²⁶, X²⁸, X²⁹, X³², X³³, X³⁵, X³⁶, X³⁷, and X³⁸.

16. The recombinant nucleic acid of claim 15 in which amino acid resides X³³, X³⁴, X³⁵, X³⁶, X³⁷, and X³⁸ are absent.

17. The recombinant nucleic acid of claim 4 in which the first polynucleotide encodes a signal peptide terminating at a signal peptidase cleavage site.

18. The recombinant nucleic acid of claim 17 in which the signal peptidase cleavage site is between amino acid residues corresponding to residues X³² and X³³.

19. The recombinant nucleic acid of claim 17 in which the signal peptide terminates at amino acid residue corresponding to residue X³².

20. The recombinant nucleic acid of claim 19 in which X³² is A.

21. The recombinant nucleic acid of any one of claims 1 to 19 in which the second polynucleotide sequence encodes a human apolipoprotein.

22. The recombinant nucleic acid of claim 21 in which the second polynucleotide sequence encodes a human apolipoprotein selected from preproapoliprotein, preproApoA I, proApoA I, ApoA I, preproApoA II, proApoA II, ApoA II, preproApoA-IV, proApoA IV, ApoA IV, ApoA V, preproApoE, proApoE, ApoE, preproApoA IMilano, proApoA IMilano, ApoA IMilano, preproApoA IParis, proApoA IParis, and ApoA IParis.

23. The recombinant nucleic acid of claim 1 in which the second polynucleotide is codon optimized for expression in a host cell.

24. The recombinant nucleic acid of claim 1 in which the first and second polynucleotides are codon optimized for expression in a host cell.

25. The recombinant nucleic acid of claim 24 which is codon optimized for expression in a Gram-positive bacteria.

26. The recombinant nucleic acid of claim 25 in which the Gram-positive bacteria is lactic acid bacteria.

27. The recombinant nucleic acid of claim 26 which is codon optimized for a lactic acid bacteria selected from the group consisting of Lactococcus spp., Streptococcus spp., Lactobacillus spp., Leuconostoc spp., Pediococcus spp., Brevibacterium spp. and Propionibacterium spp.

28. An expression vector comprising the recombinant nucleic acid of any one of claims 1 to 27.

29. The expression vector of claim 28 in which the recombinant nucleic acid is operably linked to a lactic acid bacteria control sequence.

30. The expression vector of claim 29 in which the control sequence comprises a lactic acid bacterial promoter.

31. The expression vector of claim 30 in which the lactic acid bacterial promoter comprises an inducible promoter.

32. The expression vector of claim 31 in which the inducible promoter comprises an acid inducible promoter.

33. The expression vector of claim 32 in which the acid inducible promoter is P170 promoter.

34. A host cell comprising the expression vector of claims 28 to 33.

35. The host cell of claim 34 which is a lactic acid bacterium.

36. The host cell of claim 35 in which the lactic acid bacterium is selected from Lactococcus spp., Streptococcus spp., Lactobacillus spp., Leuconostoc spp., Pediococcus spp., Brevibacterium spp. and Propionibacterium spp.

37. The host cell of claim 36 which is Lactococcus lactis.

38. The host cell of claim 36 in which the Lactococcus lactis is cremoris.

39. The host cell of claim 36 in which the Lactobacillus is Lactobacillus brevia.

40. The host cell of claim 36 in which is the Lactobacillus is Lactobacillus planetarium.

41. The host cell of claim 35 which is deficient in one or more extracellular proteases.

42. The host cell of claim 41 which is deficient in extracellular protease represented by PrtP.

43. The host cell of claim 41 which is deficient in extracellular protease represented by HtrA.

44. The host cell of claim 41 which is deficient in extracellular proteases represented by HtrA and PrtP.

45. The host cell of claim 34 in which the vector is stably integrated into the host cell chromosome.

46. The host cell of claim 34 in which the expression vector is selected from a plasmid, a transposable element, a bacteriophage, or a cosmid.

47. A method for producing apolipoprotein, comprising:
culturing the host cell of any one of claims 34 to 46 under conditions suitable for expression of the encoded apolipoprotein.

48. The method of claim 47 in which the culturing is in a liquid culture medium.

49. The method of claim 47, further comprising separating the culture medium from the host cells.

50. The method of claim 49 in which the separation is by filtration.

51. The method of claim 49 in which the separation is by centrifugation.

52. The method of claim 49 in which the separation is by electrophoresis.

53. The method of claim 47 in which the host cell is a lactic acid bacterium and the method further comprises the step of removing lactic acid from the medium.
